(19) United States
(12) Patent Application Publication
(10) Pub. No.: US 2007/0020625 A1
(43) Pub. Date: Jan. 25, 2007

(54) Sequence of the photorhabdus luminescens strain tt01 genome and uses

(76) Inventors: Eric Duchaud, Paris (FR); Sead Taourit, Aulnay-Sous-Bois (FR); Philippe Glaser, Paris (FR); Lionel Frangeul, Paris (FR); Frederik Kunst, Ivry-Sur-Seine (FR); Antoine Danchin, Paris (FR); Carmen Buchrieser, Paris (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/467,478

(22) PCT Filed: Feb. 7, 2002
(86) PCT No.: PCT/IB02/03040

§ 371 Date: Feb. 2, 2004

(30) Foreign Application Priority Data
Feb. 7, 2001 (FR) 0101659

(51) Int. Cl. C12Q 1/68; C07H 21/04

(52) U.S. Cl. 435/6; 536/23.7

(57) ABSTRACT

The present invention relates to the genomic sequence and to nucleotide sequences encoding polypeptides of Photorhabdus luminescens. The present invention further relates to polypeptides involved in operons involved in the biosynthesis of antibiotics or toxins, as well as polypeptides with activity of the antibiotic or toxin. Uses of the aforementioned polypeptides in pesticides, bactericides, or fungicides is provides. In addition, the present invention provides vectors, cells, or animals containing the sequences of the present invention.

The invention relates to the genomic sequence and to nucleotide sequences encoding polypeptides of Photorhabdus luminescens, such as polypeptides involved in operons for biosynthesis of antibiotics, or of toxins, or polypeptides with activity of the toxin or antibiotic type which can be used as a pesticide, bactericide or fungicide, and also to vectors which include said sequences, and to cells or animals transformed with these vectors.

Photorhabdus luminescens is an entomopathogenic, commensal intestinal bacterium of a nematode and insect parasite. This bacterium is both a model for studying host-parasite interactions and a bacterium which has many industrial applications because of its ability to synthesize numerous toxins (insecticides, bactericides and fungicides) and to secrete numerous enzymes.

In order to obtain an overall understanding of the genetic determinants involved in these processes, sequencing of the Photorhabdus luminescens genome was carried out.

The choice of the TT01 strain of Photorhabdus luminescens, subspecies laumondii, the sequencing of whose genome was carried out in the present invention, is very important since this strain has several advantages:
its genome is stable;
it can be cultured on a Petri dish;
it is central in the phylogenetic tree, and therefore representative of the species; and
its associated nematode is known and cultured (Heterorhabditis bacteriophora HP88, Trinidad).

The present invention thus relates to the nucleotide and polypeptide sequences of Photorhabdus luminescens strain TT01.

Thus, an object of the present invention is to disclose the sequence of the genome of Photorhabdus luminescens strain TT01, contained in the genomic library prepared from the genome of this strain and deposited with the CNCM [French National Collection of Cultures and Micro-organisms] on May 12, 2000, under the number I-2478, and of all the genes and noncoding regulatory sequences contained in said genome. Photorhabdus luminescens strain TT01 is also identified in the present application by Photorhabdus luminescens , in an interchangeable manner.

The invention also relates to novel tools for typing Photorhabdus strains. These tools might be of the DNA “chip” type or of another type. The novel characteristics of these typing tools will be as follows:
rapidity and simplicity of use;
high capacity for discriminating between strains; and
possibility of providing information on the genomic content of the strain analyzed.

The present invention therefore relates to an isolated nucleotide sequence derived from the Photorhabdus luminescens genome, characterized in that it comprises a sequence chosen from the sequences SEQ ID No. 1 to SEQ ID No. 41 and the sequences SEQ ID No. 5826 to SEQ ID No. 5834.

The sequences SEQ ID No. 1 to SEQ ID No. 41 represent the sequences of 41 contigs which altogether cover the genomic sequence of Photorhabdus luminescens TT01.

It has been possible to reassemble these sequences SEQ ID No. 1 to SEQ ID No. 41 and to smooth them back out into 9 new contigs which altogether also cover the genomic sequence of Photorhabdus luminescens TT01. The sequences SEQ ID No. 5826 to SEQ ID No. 5834 represent the sequences of these 9 contigs.

The nucleotide sequences SEQ ID No. 1 to SEQ ID No. 41 and SEQ ID No. 5826 to SEQ ID No. 5834 were obtained by sequencing the Photorhabdus luminescens TT01 genome using the “shotgun” technique (cf. examples). Despite the great precision of these sequences SEQ ID No. 1 to SEQ ID No. 41 or SEQ ID No. 5826 to SEQ ID No. 5834, it is possible that these sequences do not give a 100% perfect representation, after assembly, of the nucleotide sequence of the Photorhabdus luminescens TT01 genome, and that some rare sequencing errors or indeterminations remain in these sequences. In the present invention, the presence of an indetermination of an amino acid is denoted by “Xaa” and that of a nucleotide is denoted by “N” or “n” in the sequence listing hereinafter. These few rare errors or indeterminations may be easily demonstrated and corrected by those skilled in the art using the whole chromosome and/or its representative fragments according to the invention, and standard methods of amplification, cloning and sequencing, it being possible for the sequences obtained to be easily compared, in particular by means of computer software, and using computer-readable media for recording the sequences according to the invention, as described, for example, below. After correction of these possible rare errors or indeterminations, the corrected nucleotide sequence obtained would still comprise at least 97%, preferably at least 98%, 98.5%, 99% or 99.9%, identity with the genomic sequence obtained after assembly of these nucleotide sequences SEQ ID No. 1 to SEQ ID No. 41 or SEQ ID No. 5826 to SEQ ID No. 5834.

The present invention also relates to an isolated nucleotide sequence derived from the Photorhabdus luminescens genome, characterized in that it is chosen from:
a) a nucleotide sequence comprising at least 75%, 80%, 85%, 90%, 95%, 98% or 99% identity with a sequence chosen from the sequences SEQ ID No. 1 to SEQ ID No. 41 or SEQ ID No. 5826 to SEQ ID No. 5834;
b) a nucleotide sequence comprising a representative fragment of a sequence chosen from the sequences SEQ ID No. 1 to SEQ ID No. 41 or SEQ ID No. 5826 to SEQ ID No. 5834;
c) a nucleotide sequence complementary to a nucleotide sequence as defined in a) or b);
d) a nucleotide sequence of the RNA corresponding to one of the sequences as defined in a), b) or c);
e) a nucleotide sequence as defined in a), b), c) or
d), which has been modified;
f) a nucleotide sequence which hybridizes, under high stringency conditions, with a sequence chosen from SEQ ID No. 1 to SEQ ID No. 41 or SEQ ID No. 5826 to SEQ ID No. 5834, and which comprises at least 20 nucleotides, preferably at least 25, 30, 50, 75, 100, 150, 200, 250, 500, 750, 1 000, 1 500, 2 000 or 2 500 nucleotides.

More particularly, a subject of the present invention is also a nucleotide sequence included in one of the sequences SEQ ID No. 1 to SEQ ID No. 41 or one of the sequences SEQ ID No. 5826 to SEQ ID No. 5834, and in that it encodes a polypeptide chosen from the polypeptides of sequence SEQ ID No. 42 to SEQ ID No. 3855 or from the polypeptides encoded by a sequence SEQ ID No. 5835 to SEQ ID No. 10784.

Preferably, the polypeptides encoded by one of the sequences SEQ ID No. 5835 to SEQ ID No. 10784 are the polypeptides for which the sequence of at least 5 amino acids is obtained by taking as reading frame the first nucleotide of the sequences SEQ ID No. 5835 to SEQ ID No. 10784.

Very preferably, a subject of the invention is also a nucleotide sequence, characterized in that it encodes a polypeptide whose function annotated in table I hereinafter, final column, or in table II hereinafter, penultimate column, corresponds to an activity of the toxin and/or antibiotic type, or to an operon involved in the synthesis of a toxin and/or of an antibiotic, which polypeptide is preferably chosen from:

a) the polypeptides of sequences SEQ ID No. 61,

SEQ ID No. 62, SEQ ID No. 67, SEQ ID No. 171, SEQ ID No. 221, SEQ ID No. 268, SEQ ID No. 288, SEQ ID No. 380, SEQ ID No. 426, SEQ ID No. 438, SEQ ID No. 448, SEQ ID No. 453, SEQ ID No. 455, SEQ ID No. 456, SEQ ID No. 458, SEQ ID No. 501, SEQ ID No. 516, SEQ ID No. 530, SEQ ID No. 542, SEQ ID No. 551, SEQ ID No. 720, SEQ ID No. 761, SEQ ID No. 762, SEQ ID No. 814, SEQ ID No. 859, SEQ ID No. 860, SEQ ID No. 861, SEQ ID No. 862, SEQ ID No. 869, SEQ ID No. 1079, SEQ ID No. 1168, SEQ ID No. 1174, SEQ ID No. 1176, SEQ ID No. 1413, SEQ ID No. 1414, SEQ ID No. 1415, SEQ ID No. 1416, SEQ ID No. 1417, SEQ ID No. 1457, SEQ ID No. 1651, SEQ ID No. 1856, SEQ ID No. 1869, SEQ ID No. 2021, SEQ ID No. 2080, SEQ ID No. 2152, SEQ ID No. 2162, SEQ ID No. 2173, SEQ ID No. 2251, SEQ ID No. 2295, SEQ ID No. 2306, SEQ ID No. 2317, SEQ ID No. 2328, SEQ ID No. 2340, SEQ ID No. 2342, SEQ ID No. 2351, SEQ ID No. 2500, SEQ ID No. 3228, SEQ ID No. 3230, SEQ ID No. 3311, SEQ ID No. 3317, SEQ ID No. 3318, SEQ ID No. 3319, SEQ ID No. 3320, SEQ ID No. 3322, SEQ ID No. 3323, SEQ ID No. 3326, SEQ ID No. 3327, SEQ ID No. 3328, SEQ ID No. 3375, SEQ ID No. 3376, SEQ ID No. 3377, SEQ ID No. 3378, SEQ ID No. 3422, SEQ ID No. 3489, SEQ ID No. 3503, SEQ ID No. 3609, SEQ ID No. 3623, SEQ ID No. 3624, SEQ ID No. 3772, SEQ ID No. 3783, SEQ ID No. 3788 and SEQ ID No. 3794; or

b) the polypeptides encoded by the sequences SEQ ID No. 5835 to SEQ ID No. 10784 homologous to the sequences as defined in a) above, as indicated in the final column of table II.

These 82 polypeptides of sequence as defined in paragraph a) above, whose function is associated with an activity of the toxin or antibiotic type, or their homologous polypeptide of table II as defined in paragraph b) above, could be identified, for example, by the presence of a consensus motif associated with these functions or by the presence of sequences juxtaposing them on the genome and involved in this type of activity.

More generally, the present invention also relates to the nucleotide sequences derived from SEQ ID No. 1 to SEQ ID No. 41 or SEQ ID No. 5826 to SEQ ID No. 5834, and encoding a polypeptide of P. luminescens, such that they can be isolated from SEQ ID No. 1 to SEQ ID No. 41 or SEQ ID No. 5826 to SEQ ID No. 5834.

In addition, the nucleotide sequences characterized in that they comprise a nucleotide sequence chosen from:
a) a nucleotide sequence encoding a polypeptide chosen from the sequences SEQ ID No. 42 to SEQ ID No. 3855 or from the polypeptides encoded by the sequences SEQ ID No. 5835 to SEQ ID No. 10784, preferably from the 82 polypeptide sequences above selected for their function associated with an activity of the toxin or antibiotic type, or their homologous peptide as defined in table II, in the final column;
b) a nucleotide sequence comprising at least 75% identity with a nucleotide sequence as defined in a), preferably at least 80%, 85%, 90%, 95%, 98% or 99% identity;
c) a complementary or RNA nucleotide sequence corresponding to a sequence as defined in a) or b);
d) a nucleotide sequence of a representative fragment of a sequence as defined in a) or c); and
e) a sequence as defined in a) or c), which has been modified
   are also subjects of the invention.

The terms “nucleic acid”, “nucleic acid sequence”, “polynucleotide”, “oligonucleotide”, “polynucleotide sequence” and “nucleotide sequence”, terms which will be used indifferently in the present description, are intended to denote a precise chain of nucleotides, which may or may not be modified, making it possible to define a fragment or a region of a nucleic acid, which may or may not comprise unnatural nucleotides, and which may correspond equally to a double-stranded DNA, a single-stranded DNA and products of transcription of said DNAs. Thus, the nucleic acid sequences according to the invention also encompass PNAs (Peptide Nucleic Acids).

It should be understood that the present invention does not concern the nucleotide sequences in their natural chromosomal environment, i.e. in their natural state. They are sequences which have been isolated and/or purified, i.e. they have been taken directly or indirectly, for example by copying, their environment having been at least partially modified. The nucleic acids obtained by chemical synthesis are thus also intended to be denoted.

For the purpose of the present invention, the term “percentage identity” between two nucleic acid or amino acid sequences is intended to denote a percentage of nucleotides or of amino acid residues which are identical between the two sequences to be compared, obtained after best alignment, this percentage being purely statistical and the differences between the two sequences being distributed randomly and over their entire length. The term “best alignment” or “optimal alignment” is intended to denote the alignment for which the percentage identity determined as below is the highest. Sequence comparisons between two nucleic acid or amino acid sequences are conventionally carried out by comparing these sequences after having optimally aligned them, said comparison being carried out by segment or by “window of comparison” so as to identify and compare the local regions of sequence similarity. The optimal alignment of the sequences for the comparison can be carried out, besides manually, by means of the local homology algorithm of Smith and Waterman (1981, Ad. App. Math., 2:482), by means of the local homology algorithm of Neddleman and Wunsch (1970, J. Mol. Biol., 48:443), by means of the similarity search method of Pearson and Lipman (1988, Proc. Natl. Acad. Sci. USA, 85:2444), by means of computer programs using these algorithms (GAP, BESTFIT, BLAST P, BLAST N, FASTA and TFASTA in the Wisconsin Genetics Software Package, Genetics Computer Group, 575 Science Dr., Madison, Wis.). In order to obtain the optimal alignment, the BLAST program is preferably used, with the BLOSUM 62 matrix. The PAM or PAM250 matrices can also be used.

The percentage identity between two nucleic acid or amino acid sequences is determined by comparing these two sequences when optimally aligned, the nucleic acid or amino acid sequence to be compared possibly comprising additions or deletions with respect to the reference sequence, for optimal alignment between these two sequences. The percentage identity is calculated by determining the number of identical positions for which the nucleotide or amino acid residue is identical in the two sequences, dividing this number of identical positions by the total number of positions compared, and multiplying the result obtained by 100 so as to obtain the percentage identity between these two sequences.

The expression “nucleic acid sequences exhibiting a percentage identity of at least 75%, preferably 80%, 85%, 90%, 95%, 98% or 99%, after optimal alignment, with a reference sequence” is intended to denote the nucleic acid sequences exhibiting, compared to the reference nucleic acid sequence, certain modifications such as in particular a deletion, a truncation, an extension, a chimeric fusion and/or a substitution, in particular of the point type, and the nucleic acid sequence of which exhibits at least 75%, preferably 80%, 85%, 90%, 95%, 98% or 99%, identity, after optimal alignment, with the reference nucleic acid sequence. They are preferably sequences for which the complementary sequences are capable of hybridizing specifically with the reference sequences. Preferably, the specific or high stringency hybridization conditions will be such that they provide at least 75%, preferably 80%, 85%, 90%, 95%, 98% or 99%, identity, after optimal alignment, between one of the two sequences and the sequence complementary thereto.

A hybridization under high stringency conditions means that the conditions of temperature and of ionic strength are chosen such that they make it possible to maintain the hybridization between two complementary DNA fragments. By way of illustration, high stringency conditions for the hybridization step for the purposes of defining the polynucleotide fragments described above are advantageously as follows.

The DNA-DNA or DNA-RNA hybridization is carried out in two steps: (1) prehybridization at 42° C. for 3 hours in phosphate buffer (20 mM, pH 7.5) containing 5×SSC (1×SSC corresponds to a solution of 0.15M NaCl+0.015M sodium citrate), 50% of formamide, 7% of sodium dodecyl sulfate (SDS), 10× Denhardt's, 5% of dextran sulfate and 1% of salmon sperm DNA; (2) hybridization per se for 20 hours at a temperature which depends on the length of the probe (i.e.: 42° C. for a probe>100 nucleotides in length) followed by 2 washes of 20 minutes at 20° C. in 2×SSC+2% SDS and 1 wash of 20 minutes at 20° C. in 0.1×SSC+0.1% SDS. The final wash is carried out in 0.1×SSC+0.1% SDS for 30 minutes at 60° C. for a probe>100 nucleotides in length. The high stringency hybridization conditions described above for a polynucleotide of defined length can be adjusted by those skilled in the art for longer or shorter oligonucleotides, according to the teaching of Sambrook et al. (1989, Molecular cloning: a laboratory manual. 2nd Ed. Cold Spring Harbor).

In addition, the expression “representative fragment of sequences according to the invention” is intended to denote any nucleotide fragment having at least 15 consecutive nucleotides, preferably at least 20, 25, 30, 50, 75, 100, 150, 200, 300 and 450 consecutive nucleotides, of the sequence from which it is derived.

The term “representative fragment” is intended to mean in particular a nucleic acid sequence encoding a biologically active fragment of a polypeptide, as defined later.

The term “representative fragment” is also intended to mean the intergenic sequences, and in particular the nucleotide sequences carrying the regulatory signals (promoters, terminators, or even enhancers, etc).

Among said representative fragments, preference is given to those having nucleotide sequences corresponding to open reading frames, referred to as ORF sequences, included in general between an initiation codon and a stop codon, or between two stop codons, and encoding polypeptides, preferably with at least 100 amino acids, such as, for example, without being limited thereto, the ORF sequences which will subsequently be described.

The numbering of the ORF nucleotide sequences which will subsequently be used in the present description corresponds to the numbering of the amino acid sequences of the proteins encoded by said ORFs for the sequences SEQ ID No. 42 to SEQ ID No. 3855. The numbering of the ORF nucleotide sequences SEQ ID No. 5835 to SEQ ID No. 10784 will subsequently be used in the present description for the numbering of the amino acid sequences of the proteins encoded by said ORFs SEQ ID No. 5835 to SEQ ID No. 10784.

The representative fragments according to the invention can be obtained, for example, by specific amplification such as PCR or after digestion, with suitable restriction enzymes, of nucleotide sequences according to the invention, this method being described in particular in the work by Sambrook et al. Said representative fragments can also be obtained by chemical synthesis when they are not too long, according to methods well known to those skilled in the art.

The sequences containing sequences of the invention, or representative fragments, are also intended to include the sequences which are naturally framed by sequences which exhibit at least 75%, 80%, 85%, 90%, 95%, 98% or 99% identity with the sequences according to the invention.

The term “modified nucleotide sequence” is intended to mean any nucleotide sequence obtained by mutagenesis according to techniques well known to those skilled in the art, and comprising modifications compared to the normal sequences, preferably at most 10% of modified nucleotides compared to these normal sequences, for example mutations in the regulatory and/or promoter sequences for expression of the polypeptide, in particular leading to a modification of the level of expression or of the activity of said polypeptide.

The term “modified nucleotide sequence” is also intended to mean any nucleotide sequence encoding a modified polypeptide as defined below.

The representative fragments according to the invention can also be probes or primers, which can be used in methods for detecting, identifying, assaying or amplifying nucleic acid sequences.

For the purpose of the invention, a probe or primer is defined as being a single-stranded nucleic acid fragment or a denatured double-stranded fragment comprising, for example, from 12 bases to a few kb, in particular from 15 to a few hundred bases, preferably from 15 to 50 or 100 bases, and having a specificity of hybridization under given conditions so as to form a hybridization complex with a target nucleic acid.

The probes and primers according to the invention can be labeled directly or indirectly with a radioactive or nonradioactive compound using methods well known to those skilled in the art, in order to obtain a detectable and/or quantifiable signal (patent FR 78 10975 and bDNA of Chiron EP 225 807 and EP 510 085).

The unlabeled sequences of polynucleotides according to the invention can be used directly as a probe or primer.

The sequences are generally labeled to obtain sequences which can be used for many applications. The labeling of the primers or of the probes according to the invention is carried out with radioactive elements or with nonradioactive molecules.

Among the radioactive isotopes used, mention may be made of ³²P, ³³P ³⁵S, ³H or ¹²⁵I. The nonradioactive entities are selected from ligands such as biotin, avidin, streptavidin or digoxigenin, haptens, dyes, and luminescent agents such as radioluminescent, chemiluminescent, bioluminescent, fluorescent or phosphorescent agents.

The polynucleotides according to the invention can thus be used as a primer and/or a probe in methods using in particular the PCR (polymerase chain reaction) technique (Rolfs et al., 1991, Berlin: Springer-Verlag). This technique requires choosing pairs of oligonucleotide primers framing the fragment which must be amplified. Reference may, for example, be made to the technique described in U.S. Pat. No. 4,683,202. The amplified fragments can be identified, for example after agarose or polyacrylamide gel electrophoresis, or after a chromatography technique such as gel filtration or ion exchange chromatography, and then sequenced. The specificity of the amplification can be controlled using the nucleotide sequences of polynucleotides of the invention as a matrix, plasmids containing these sequences or else the derived amplification products. The amplified nucleotide fragments can be used as reagents in hybridization reactions in order to demonstrate the presence, in a biological sample, of a target nucleic acid of sequence complementary to that of said amplified nucleotide fragments.

The invention is also directed toward the nucleic acids which can be obtained by amplification using primers according to the invention.

Other techniques for amplifying the target nucleic acid can advantageously be employed as an alternative to PCR (PCR-like), using a pair of primers of nucleotide sequences according to the invention. The term “PCR-like” is intended to denote all the methods using direct or indirect reproductions of nucleic acid sequences, or else in which the labeling systems have been amplified; these techniques are, of course, known. In general, they involve amplification of the DNA with a polymerase; when the sample of origin is an RNA, a reverse transcription should be carried out beforehand. A very large number of methods currently exist for this amplification, such as, for example, the SDA (strand displacement amplification) technique (Walker et al., 1992, Nucleic Acids Res., 20:1691), the TAS (transcription-based amplification system) technique described by Kwoh et al. (1989, Proc. Natl. Acad. Sci. USA, 86, 1173), the 3SR (self-sustained sequence replication) technique described by Guatelli et al. (1990, Proc. Natl. Acad. Sci. USA, 87: 1874), the NASBA (nucleic acid sequence based amplification) technique described by Kievitis et al. (1991, J. Virol. Methods, 35, 273), the TMA (transcription mediated amplification) technique, the LCR (ligase chain reaction) technique described by Landegren et al. (1988, Science, 241, 1077), the RCR (repair chain reaction) technique described by Segev (1992, Kessler C. Springer Verlag, Berlin, N.Y., 197-205), the CPR (cycling probe reaction) technique described by Duck et al. (1990, Biotechniques, 9, 142) and the Q-beta-replicase amplification technique described by Miele et al. (1983, J. Mol. Biol., 171, 281). Some of these techniques have since been improved.

When the target polynucleotide to be detected is an mRNA, an enzyme of the reverse transcriptase type is advantageously used, prior to carrying out an amplification reaction using the primers according to the invention, or to carrying out a method of detection using the probes of the invention, in order to obtain a cDNA from the mRNA contained in the biological sample.

The cDNA obtained will then serve as a target for the primers or the probes used in the method of amplification or of detection according to the invention.

The probe hybridization technique can be carried out in various ways (Matthews et al., 1988, Anal. Biochem., 169, 1-25). The most general method consists in immobilizing the nucleic acid, extracted from the cells of various tissues or from cells in culture, on a support (such as nitrocellulose, nylon or polystyrene) and in incubating, under well-defined conditions, the immobilized target nucleic acid with the probe. After hybridization, the excess probe is removed and the hybrid molecules formed are detected by the appropriate method (measurement of the radioactivity, of the fluorescence or of the enzyme activity associated with the probe).

According to another embodiment of the nucleic acid probes according to the invention, the latter can be used as capture probes. In this case, a probe, termed “capture probe”, is immobilized on a support and is used to capture, by specific hybridization, the target nucleic acid obtained from the biological sample to be tested, and the target nucleic acid is then detected using a second probe, termed “detection probe”, labeled with a readily detectable element.

Among the advantageous nucleic acid fragments, mention should thus in particular be made of antisense oligonucleotides, i.e. oligonucleotides whose structure provides, by hybridization with the target sequence, inhibition of the expression of the corresponding product. Mention should also be made of sense oligonucleotides which, by interaction with proteins involved in regulating the expression of the corresponding product, will induce either inhibition or activation of this expression.

Preferably, the probes or primers according to the invention are immobilized on a support in a covalent or noncovalent manner. In particular, the support can be a DNA chip or a high or medium density filter, also a subject of the present invention (patents WO 97/29212, WO 98/27317, WO 97/10365 and WO 92/10588).

The term “DNA chip” or “high density filter” is intended to denote a support on which are attached DNA sequences, it being possible to pinpoint each one of them by its geographical location. These chips or filters differ mainly by their size, the material of the support and, optionally, the number of DNA sequences which are attached thereto.

The probes or primers according to the first invention can be attached to solid supports, in particular the DNA chips, by various methods of production. In particular, in situ synthesis can be carried out by photochemical addressing or by inkjet. Other techniques consist in carrying out an ex situ synthesis and attaching the probes to the support of the DNA chip by mechanical or electronic addressing or by inkjet. These various methods are well known to those skilled in the art.

A nucleotide sequence (probe or primer) according to the invention therefore makes it possible to detect and/or amplify specific nucleic acid sequences. In particular, the detection of these said sequences is facilitated when the probe is attached to a DNA chip or to a high density filter.

The use of DNA chips or of high density filters in fact makes it possible to determine the gene expression in an organism having a genomic sequence close to P. luminescens, and to type the strain in question.

The genomic sequence of P. luminescens, supplemented by the identification of the genes of these organisms, as presented in the present invention, serves as a basis for constructing these DNA chips or filters.

The preparation of these filters or chips consists in synthesizing oligonucleotides, corresponding to the 5′ and 3′ ends of the genes or to more internal fragments, in order to amplify fragments of an appropriate length, for example of between approximately 300 and 800 bases. These oligonucleotides are chosen using the genomic sequence and its annotations disclosed in the present invention. The temperature for pairing these oligonucleotides at the corresponding places on the DNA should be approximately the same for each oligonucleotide. This makes it possible to prepare DNA fragments corresponding to each gene using appropriate PCR conditions in a highly automated environment. The amplified fragments are then immobilized on filters or supports made of glass, silicon or synthetic polymers and these media are used for the hybridization.

The availability of such filters and/or chips and of the corresponding annotated genomic sequence makes it possible to study the expression of large sets, or even all, of the genes of Photorhabdus luminescens , in particular of P. luminescens TT01, by preparing the complementary DNAs and hybridizing them to the DNA or to the oligonucleotides immobilized on the filters or the chips. Similarly, the filters and/or the chips make it possible to study the strain or species variability by preparing the DNA of these organisms and hybridizing them to the DNA or to the oligonucleotides immobilized on the filters or the chips.

The differences between the genomic sequences of the various strains or species can greatly affect the intensity of the hybridization and, consequently, disturb the interpretation of the results. It may therefore be necessary to have the precise sequence of the genes of the strain intended to be studied. The method for detecting the genes described later in detail, involving determining the sequence of random fragments of a genome and organizing them according to the sequence of the genome of P. luminescens, in particular of P. luminescens TT01, disclosed in the present invention, may be very useful.

The nucleotide sequences according to the invention can be used in DNA chips to carry out mutation analysis. This analysis is based on constituting chips capable of analyzing each base of a nucleotide sequence according to the invention. For this purpose, use may in particular be made of the techniques of microsequencing on a DNA chip. The mutations are detected by extension of immobilized primers which hybridize to the matrix of the analyzed sequences, just at a position adjacent to that of the mutated nucleotide sought. A single-stranded, RNA or DNA matrix of the sequences to be analyzed will advantageously be prepared according to conventional methods, using products amplified according to PCR-type techniques. The single-stranded DNA or RNA matrices thus obtained are then deposited onto the DNA chip, under conditions which allow their specific hybridization to the immobilized primers. A thermostable polymerase, for example Tth or Taq DNA polymerase, specifically extends the 3′ end of the immobilized primer with a labeled nucleotide analog complementary to the nucleotide at the position of the variable site; for example, thermocycling is carried out in the presence of fluorescent dideoxyribonucleotides. The experimental conditions will be adjusted in particular to the chips used, to the immobilized primers, to the polymerases used and to the labeling system chosen. One advantage of microsequencing, compared to techniques based on probe hybridization, is that it makes it possible to identify all the variable nucleotides with optimal discrimination under homogeneous reaction conditions; when used on DNA chips, it allows optimal resolution and specificity for the routine and industrial detection of mutations in a multiplex.

A DNA chip or a filter can be an extremely advantageous tool for determining, detecting and/or identifying a microorganism. Thus, the DNA chips according to the invention which also contain at least one nucleotide sequence of a microorganism other than Photorhabdus luminescens, immobilized on the support of said chip, are also preferred. The microorganism chosen is preferably done so from the bacteria of the genus Photorhabdus (hereinafter referred to as P. luminescens-related bacteria), or the variants of Photorhabdus luminescens TT01.

A DNA chip or a filter according to the invention is a very useful element of certain kits or sets for detecting and/or identifying microorganisms, in particular bacteria belonging to the species Photorhabdus luminescens , which are also the subject of the invention.

Moreover, the DNA chips or the filters according to the invention, containing probes or primers specific for Photorhabdus luminescens , are very advantageous elements of kits or sets for detecting and/or quantifying the expression of Photorhabdus luminescens genes.

Specifically, the control of gene expression is a critical point for optimizing the growth and yield of a strain, either by allowing the expression of one or more new genes, or by modifying the expression of genes already present in the cell. The present invention provides all of the sequences naturally active in P. luminescens which allow gene expression. It thus makes it possible to determine all the sequences expressed in P. luminescens. It also provides a tool for pinpointing the genes whose expression follows a given pattern. To do this, the DNA of all or some of the genes of P. luminescens can be amplified using primers according to the invention, and then attached to a support such as, for example, glass or nylon or a DNA chip, in order to construct a tool for following the expression profile of these genes. This tool, consisting of this support containing the coding sequences, serves as a matrix of hybridization to a mixture of labeled molecules reflecting the messenger RNAs expressed in the cell (in particular the labeled probes according to the invention). By repeating this experiment at various moments and combining all of these data using suitable processing, the expression profiles of all these genes are then obtained. The knowledge of the sequences which follow a given regulatory scheme can also be exploited to search for, in a directed manner, for example by homology, other sequences following, overall, but in a slightly different way, the same regulatory scheme. In addition, it is possible to isolate each control sequence present upstream of the segments acting as probes and to follow the activity thereof using a suitable means such as a reporter gene (luciferase, β-galactosidase, GFP). These isolated sequences can then be modified and assembled by metabolic engineering with sequences of interest with a view to the optimal expression thereof.

The invention also relates to the polypeptides encoded by a nucleotide sequence according to the invention, preferably by a representative fragment of the preceding sequences, corresponding to an ORF sequence. In particular, the polypeptides of Photorhabdus luminescens TT01 of SEQ ID No. 42 to SEQ ID No. 3855 or encoded by SEQ ID No. 5835 to SEQ ID No. 10784 are a subject of the invention.

The invention also comprises the polypeptides characterized in that they comprise a polypeptide chosen from:
a) a polypeptide of sequence SEQ ID No. 42 to SEQ ID No. 3855 or encoded by a sequence SEQ ID No. 5835 to SEQ ID No. 10784;
b) a polypeptide exhibiting at least 80%, preferably 85%, 90%, 95% and 98%, identity with a polypeptide according to the invention;
c) a fragment of at least 5 amino acids of a polypeptide as defined in a);
d) a biologically active fragment of a polypeptide as defined in a); and
e) a polypeptide as defined in a), b), c) or d), which has been modified.

The nucleotide sequences encoding the polypeptides described above are also a subject of the invention.

In the present description, the terms “polypeptides”, “polypeptide sequences”, “peptides” and “proteins” are interchangeable. The term “polypeptide” comprises any amino acid sequence making it possible to generate an antibody response.

It should be understood that the invention does not concern the polypeptides in natural form, i.e. they are not taken in their natural environment. On the other hand, it concerns those which it has been possible to isolate or obtain by purification from natural sources, or else those obtained by genetic recombination or by chemical synthesis, and they can then comprise unnatural amino acids as will be described below.

The expression “polypeptide exhibiting a certain percentage identity with another”, for which the expression “homologous polypeptide” will also be used, is intended to denote the polypeptides exhibiting, compared to the natural polypeptides, certain modifications, in particular a deletion, addition or substitution of at least one amino acid, a truncation, an extension, a chimeric solution and/or a mutation, or the polypeptides exhibiting post-translational modifications. Among the homologous polypeptides, preference is given to those in which the amino acid sequence exhibits at least 80%, preferably 85%, 90%, 95%, 98% or 99%, identity, after optimal alignment, with the amino acid sequences of the polypeptides according to the invention. In the case of a substitution, one or more consecutive or nonconsecutive amino acid(s) may be replaced with “equivalent” amino acids. The expression “equivalent amino acids” is here aimed at denoting any amino acid capable of being substituted for one of the amino acids of the basic structure without, however, essentially modifying the biological activities of the corresponding peptides as they will be defined subsequently.

These equivalent amino acids can be determined either based on their structural homology with the amino acids for which they substitute, or based on results of comparative biological activity assays between the various polypeptides liable to be produced.

By way of example, mention is made of the substitution possibilities which can be made without this resulting in a profound modification of the biological activity of the corresponding modified polypeptide. It is thus possible to replace leucine with valine or isoleucine, aspartic acid with glutamic acid, glutamine with asparagine, arginine with lysine, etc, it naturally being possible to envision the reverse substitutions under the same conditions.

The homologous polypeptides also correspond to the polypeptides encoded by the nucleotide sequences which exhibit a certain percentage identity with the nucleotide sequences of the invention or which are identical, as previously defined, and thus comprise, in the present definition, mutated polypeptides or polypeptides corresponding to inter- or intraspecies variations which can exist in Photorhabdus, and which correspond in particular to truncations, substitutions, deletions and/or additions of at least one amino acid residue.

It is understood that the percentage identity between two polypeptides is calculated in the same way as between two nucleic acid sequences. Thus, the percentage identity between two polypeptides is calculated, after optimal alignment of these two sequences, on a window of maximum homology. To define said window of maximum homology, the same algorithms as for the nucleic acid sequences can be used.

The expression “biologically active fragment of a polypeptide according to the invention” is intended to denote in particular a polypeptide fragment, as defined below, exhibiting at least one of the biological characteristics of the polypeptides according to the invention, in particular in that it is capable of exerting, in general, even a partial activity, such as, for example:
an enzymatic (metabolic) activity or an activity which may be involved in the biosynthesis or the biodegradation of organic or inorganic compounds, or preferably a toxic or antibiotic activity, in particular for insects or microorganisms (bacteria or fungi), or else an activity involved in the biosynthesis of these toxins or antibiotics; such proteins with enzymatic activity may in particular be used in methods for screening and/or selecting compounds capable of modifying this activity, in particular of inhibiting it;
a structural activity (cell envelope, chaperone molecule, ribosome). Proteins corresponding especially to extramembrane proteins may in particular be used as an immunogen for producing mono- or polyclonal antibodies directed specifically against these extramembrane proteins;
a transport activity (energy transport, ion transport); or an activity in protein secretion;
an activity in the process of replication, amplification, preparation, transcription, translation or maturation, in particular of DNA, of RNA or of proteins.

The expression “polypeptide fragment according to the invention” is intended to denote a polypeptide comprising at least 5 amino acids, preferably 10, 15, 25, 50, 100 and 150 amino acids.

The polypeptide fragments can correspond to isolated or purified fragments naturally present in the strains of Photorhabdus, or to fragments which can be obtained by cleavage of said polypeptide with a proteolytic enzyme, such as trypsin or chymotrypsin or collagenase, or with a chemical reagent (cyanogen bromide, CNBr) or by placing said polypeptide in a very acidic environment (for example at pH=2.5). Polypeptide fragments can also be prepared by chemical synthesis, from hosts transformed with an expression vector according to the invention which contain a nucleic acid which allows expression of said fragment and is placed under the control of the appropriate regulatory and/or expression elements.

The term “modified polypeptide” of a polypeptide according to the invention is intended to denote a polypeptide obtained by genetic recombination or by chemical synthesis as described later, which exhibits at least one modification compared to the normal sequence, and preferably at most 10% of modified amino acids compared to the normal sequence. These modifications may in particular be made on amino acids necessary for the specificity or the effectiveness of the activity, or responsible for the structural conformation, for the charge or for the hydrophobicity of the polypeptide according to the invention. It is thus possible to create polypeptides with equivalent, increased or decreased activity, or with equivalent, stricter or broader specificity. Among the modified polypeptides, mention should be made of the polypeptides in which up to five amino acids can be modified, truncated at the N- or C-terminal end, or else deleted, or added.

As is indicated, the aim of the modifications to a polypeptide is in particular:
to allow its use in methods of biosynthesis or of biodegradation of organic or inorganic compounds, or in the biosynthesis of toxins or of antibiotics,
to allow its use in methods of replication, of amplification, of repair and of regulation of transcription, translation or maturation in particular of DNA, RNA or proteins; to allow its enhanced secretion,
to modify its solubility, or the effectiveness or specificity of its activity, or else to facilitate its purification.

Chemical synthesis also has the advantage of being able to use unnatural amino acids or nonpeptide bonds. Thus, it may be advantageous to use unnatural amino acids, for example in the D form, or amino acid analogs, in particular sulfur-containing forms.

The invention also relates to the nucleic acid or peptide sequences according to the present invention with the exception of the nucleic acid or peptide sequences described in documents WO 99/54472, WO 99/42589, WO 99/03328, WO 98/08932 and EP 0 823 215.

The present invention provides the nucleotide sequence of the Photorhabdus luminescens TT01 genome in the form of 41 contigs or in the form of 9 contigs, and also some polypeptide sequences.

The nucleic acid or peptide sequences below, characterized by their function, can also be identified by their nucleotide and amino acid sequence with reference to table I.

Preferably, the invention relates to a nucleotide sequence according to the invention, characterized in that it encodes a polypeptide of Photorhabdus luminescens TT01 with activity of the toxin and/or antibiotic type, or involved in the synthesis of these toxins and/or antibiotics.

Preferably, the invention relates to a nucleotide sequence according to the invention, characterized in that it encodes a polypeptide of Photorhabdus luminescens TT01, or one of its fragments, involved in amino acid biosynthesis.

Preferably, the invention relates to a nucleotide sequence according to the invention, characterized in that it encodes a polypeptide of Photorhabdus luminescens TT01, or one of its fragments, involved in the biosynthesis of cofactors, prosthetic groups and transporters.

Preferably, the invention relates to a nucleotide sequence according to the invention, characterized in that it encodes a cell envelope polypeptide or a polypeptide present at the surface of Photorhabdus luminescens TT01, or one of its fragments.

Preferably, the invention relates to a nucleotide sequence according to the invention, characterized in that it encodes a polypeptide of Photorhabdus luminescens TT01, or one of its fragments, involved in the cellular machinery.

Preferably, the invention relates to a nucleotide sequence according to the invention, characterized in that it encodes a polypeptide of Photorhabdus luminescens TT01, or one of its fragments, involved in central intermediate metabolism.

Preferably, the invention relates to a nucleotide sequence according to the invention, characterized in that it encodes a polypeptide of Photorhabdus luminescens TT01, or one of its fragments, involved in energy metabolism.

Preferably, the invention relates to a nucleotide sequence according to the invention, characterized in that it encodes a polypeptide of Photorhabdus luminescens TT01, or one of its fragments, involved in fatty acid and phospholipid metabolism.

Preferably, the invention relates to a nucleotide sequence according to the invention, characterized in that it encodes a polypeptide of Photorhabdus luminescens TT01, or one of its fragments, involved in the metabolism of nucleotides, purines, pyrimidines or nucleosides.

Preferably, the invention relates to a nucleotide sequence according to the invention, characterized in that it encodes a polypeptide of Photorhabdus luminescens TT01, or one of its fragments, involved in regulatory functions.

Preferably, the invention relates to a nucleotide sequence according to the invention, characterized in that it encodes a polypeptide of Photorhabdus luminescens TT01, or one of its fragments, involved in the replication process.

Preferably, the invention relates to a nucleotide sequence according to the invention, characterized in that it encodes a polypeptide of Photorhabdus luminescens TT01, or one of its fragments, involved in the transcription process.

Preferably, the invention relates to a nucleotide sequence according to the invention, characterized in that it encodes a polypeptide of Photorhabdus luminescens TT01, or one of its fragments, involved in the translation process.

Preferably, the invention relates to a nucleotide sequence according to the invention, characterized in that it encodes a polypeptide of Photorhabdus luminescens TT01, or one of its fragments, involved in the process of protein transport and binding.

Preferably, the invention relates to a nucleotide sequence according to the invention, characterized in that it encodes a polypeptide of Photorhabdus luminescens TT01, or one of its fragments, involved in adaptation to atypical conditions.

Preferably, the invention relates to a nucleotide sequence according to the invention, characterized in that it encodes a polypeptide of Photorhabdus luminescens TT01, or one of its fragments, [lacuna] in sensitivity to medicinal products and analogs.

Preferably, the invention relates to a nucleotide sequence according to the invention, characterized in that it encodes a polypeptide of Photorhabdus luminescens TT01, or one of its fragments, involved in functions relating to transposons.

Preferably, the invention relates to a nucleotide sequence according to the invention, characterized in that it encodes a polypeptide specific for Photorhabdus luminescens TT01, or one of its fragments.

In another aspect, a subject of the invention is preferably a polypeptide according to the invention, characterized in that it is a polypeptide of Photorhabdus luminescens TT01, or one of its fragments, [lacuna] activity of the toxin and/or antibiotic type, or involved in the synthesis of these toxins and/or antibiotics.

In another aspect, a subject of the invention is preferably a polypeptide according to the invention, characterized in that it is a polypeptide of Photorhabdus luminescens TT01, or one of its fragments, involved in amino acid biosynthesis.

In another aspect, a subject of the invention is preferably a polypeptide according to the invention, characterized in that it is a polypeptide of Photorhabdus luminescens TT01, or one of its fragments, involved in the biosynthesis of cofactors, prosthetic groups and transporters.

In another aspect, a subject of the invention is preferably a polypeptide according to the invention, characterized in that it is a cell envelope polypeptide or a surface polypeptide of Photorhabdus luminescens TT01, or one of its fragments.

In another aspect, a subject of the invention is preferably a polypeptide according to the invention, characterized in that it is a polypeptide of Photorhabdus luminescens TT01, or one of its fragments, involved in the cellular machinery.

In another aspect, a subject of the invention is preferably a polypeptide according to the invention, characterized in that it is a polypeptide of Photorhabdus luminescens TT01, or one of its fragments, involved in central intermediate metabolism.

In another aspect, a subject of the invention is preferably a polypeptide according to the invention, characterized in that it is a polypeptide of Photorhabdus luminescens TT01, or one of its fragments, involved in energy metabolism.

In another aspect, a subject of the invention is preferably a polypeptide according to the invention, characterized in that it is a polypeptide of Photorhabdus luminescens TT01, or one of its fragments, involved in fatty acid and phospholipid metabolism.

In another aspect, a subject of the invention is preferably a polypeptide according to the invention, characterized in that it is a polypeptide of Photorhabdus luminescens TT01, or one of its fragments, involved in the metabolism of nucleotides, purines, pyrimidines or nucleosides.

In another aspect, a subject of the invention is preferably a polypeptide according to the invention, characterized in that it is a polypeptide of Photorhabdus luminescens TT01, or one of its fragments, involved in regulatory functions.

In another aspect, a subject of the invention is preferably a polypeptide according to the invention, characterized in that it is a polypeptide of Photorhabdus luminescens TT01, or one of its fragments, involved in the replication process.

In another aspect, a subject of the invention is preferably a polypeptide according to the invention, characterized in that it is a polypeptide of Photorhabdus luminescens TT01, or one of its fragments, involved in the transcription process.

In another aspect, a subject of the invention is preferably a polypeptide according to the invention, characterized in that it is a polypeptide of Photorhabdus luminescens TT01, or one of its fragments, involved in the translation process.

In another aspect, a subject of the invention is preferably a polypeptide according to the invention, characterized in that it is a polypeptide of Photorhabdus luminescens TT01, or one of its fragments, involved in the process of protein transport and binding.

In another aspect, a subject of the invention is preferably a polypeptide according to the invention, characterized in that it is a polypeptide of Photorhabdus luminescens TT01, or one of its fragments, involved in adaptation to atypical conditions.

In another aspect, a subject of the invention is preferably a polypeptide according to the invention, characterized in that it is a polypeptide of Photorhabdus luminescens TT01, or one of its fragments, [lacuna] in sensitivity to medicinal products and analogs.

In another aspect, a subject of the invention is preferably a polypeptide according to the invention, characterized in that it is a polypeptide of Photorhabdus luminescens TT01, or one of its fragments, involved in functions relating to transposons.

In another aspect, a subject of the invention is preferably a polypeptide according to the invention, characterized in that it is a polypeptide specific for Photorhabdus luminescens TT01, or one of its fragments.

A subject of the invention is also the operons involved in the synthesis of antibiotics and/or of toxins.

Table I provides the list of some polypeptides according to the invention, and also their location in the contigs represented by SEQ ID No. 1 to SEQ ID No. 41, and the similarities observed after comparison in the databases.

Table II provides the list of some polypeptides according to the invention, and also their location in the contigs represented by SEQ ID No. 5826 to SEQ ID No. 5834, and the similarities observed after comparison in the databases. In table II, contigs 1 to 9 are identified by the sequences SEQ ID No. 5826 to SEQ ID No. 5834.

Entirely preferably, a subject of the invention is also the polypeptides whose functions annotated in table I, final column, or whose functions annotated in table II, penultimate column, correspond to activities of the toxin and/or antibiotic type, or to polypeptides involved in the synthesis of these toxins and/or antibiotics, which polypeptides are preferably chosen from:

a) the polypeptides of sequence SEQ ID No. 61,

SEQ ID No. 62, SEQ ID No. 67, SEQ ID No. 171, SEQ ID No. 221, SEQ ID No. 268, SEQ ID No. 288, SEQ ID No. 380, SEQ ID No. 426, SEQ ID No. 438, SEQ ID No. 448, SEQ ID No. 453, SEQ ID No. 455, SEQ ID No. 456, SEQ ID No. 458, SEQ ID No. 501, SEQ ID No. 516, SEQ ID No. 530, SEQ ID No. 542, SEQ ID No. 551, SEQ ID No. 720, SEQ ID No. 761, SEQ ID No. 762, SEQ ID No. 814, SEQ ID No. 859, SEQ ID No. 860, SEQ ID No. 861, SEQ ID No. 862, SEQ ID No. 869, SEQ ID No. 1079, SEQ ID No. 1168, SEQ ID No. 1174, SEQ ID No. 1176, SEQ ID No. 1413, SEQ ID No. 1414, SEQ ID No. 1415, SEQ ID No. 1416, SEQ ID No. 1417, SEQ ID No. 1457, SEQ ID No. 1651, SEQ ID No. 1856, SEQ ID No. 1869, SEQ ID No. 2021, SEQ ID No. 2080, SEQ ID No. 2152, SEQ ID No. 2162, SEQ ID No. 2173, SEQ ID No. 2251, SEQ ID No. 2295, SEQ ID No. 2306, SEQ ID No. 2317, SEQ ID No. 2328, SEQ ID No. 2340, SEQ ID No. 2342, SEQ ID No. 2351, SEQ ID No. 2500, SEQ ID No. 3228, SEQ ID No. 3230, SEQ ID No. 3311, SEQ ID No. 3317, SEQ ID No. 3318, SEQ ID No. 3319, SEQ ID No. 3320, SEQ ID No. 3322, SEQ ID No. 3323, SEQ ID No. 3326, SEQ ID No. 3327, SEQ ID No. 3328, SEQ ID No. 3375, SEQ ID No. 3376, SEQ ID No. 3377, SEQ ID No. 3378, SEQ ID No. 3422, SEQ ID No. 3489, SEQ ID No. 3503, SEQ ID No. 3609, SEQ ID No. 3623, SEQ ID No. 3624, SEQ ID No. 3772, SEQ ID No. 3783, SEQ ID No. 3788 and SEQ ID No. 3794;

or

b) the polypeptides encoded by the sequences SEQ ID No. 5835 to SEQ ID No. 10784, homologous to the sequences as defined in a), as indicated in the final column of table II.

The subject of the present invention is also the nucleotide and/or polypeptide sequences according to the invention, characterized in that said sequences are recorded on a recording medium, the form and nature of which facilitate the reading, analysis and/or exploitation of said sequence(s). These media may also contain other information extracted from the present invention, in particular the similarities with already known sequences, and/or information concerning the nucleotide and/or polypeptide sequences of a cell of a plant, of an animal or of a microorganism other than P. luminescens, in particular a cell or microorganism sensitive to a toxin or an antibiotic produced by P. luminescens, a bacterium of the genus Photorhabdus, or a variant of P. luminescens, in order to facilitate the comparative analysis and the exploitation of the results obtained.

Among these said recording media, preference is given in particular to computer-readable media, such as magnetic, optical, electrical or hybrid media, in particular computer disks, CD-ROMs and computer servers. Such recording media are also a subject of the invention.

The recording media according to the invention, with the information provided, are very useful for choosing nucleotide primers or probes for determining genes in Photorhabdus luminescens TT01 or strains related to this organism. Similarly, the use of these media for studying the genetic polymorphism of strains related to Photorhabdus luminescens TT01, in particular by determining the regions of colinearity, is very useful insofar as these media provide not only the nucleotide sequence of the Photorhabdus luminescens TT01 genome, but also the genomic organization in said sequence. Thus, the uses of recording media according to the invention are also subjects of the invention.

The analysis of homology between various sequences is in fact advantageously performed using sequence comparison programs, such as the Blast program, or the programs of the GCG package, described above.

The invention is also directed toward the cloning and/or expression vectors which contain a nucleotide sequence according to the invention.

The vectors according to the invention preferably comprise elements which allow the expression and/or the secretion of the nucleotide sequences in a given host cell.

The vector should then comprise a promoter, translation initiation and termination signals, and also regions suitable for regulating transcription. It must be possible for it to be maintained stably in the host cell and it may optionally contain particular signals which specify secretion of the translated protein. These various elements are chosen and optimized by those skilled in the art as a function of the cellular host used. To this effect, the nucleotide sequences according to the invention can be inserted into vectors which replicate autonomously in the host chosen, or may be vectors which integrate in the host chosen.

Such vectors are prepared by methods commonly used by those skilled in the art, and the resulting clones can be introduced into a suitable host by standard methods, such as lipofection, electroporation, heat shock or chemical methods.

The vectors according to the invention are, for example, vectors of plasmid or viral origin. They are useful for transforming host cells in order to clone or express the nucleotide sequences according to the invention.

Among these vectors, preference is also given to the cloning and/or expression vectors according to the invention, characterized in that they contain a nucleotide sequence chosen from the sequences SEQ ID No. 3856 to SEQ ID No. 5825, and SEQ ID No. 5835 to SEQ ID No. 10784, or their fragment derived from the P. luminescens genome, in particular the sequences encoding the polypeptides with toxin or antibiotic activity or involved in these activities, in particular those mentioned above whose functions annotated in table I below correspond to these activities.

The invention also comprises the host cells transformed with a vector according to the invention.

The cellular host may be chosen from prokaryotic or eukaryotic systems, for example bacterial cells, but also yeast cells or animal cells, in particular mammalian cells. Insect cells or plant cells may also be used. The preferred host cells according to the invention are in particular prokaryotic cells, preferably bacteria belonging to the genus Photorhabdus or to the species Photorhabdus luminescens , more particularly Photorhabdus luminescens TT01.

The invention also relates to the plants and animals, except humans, which comprise a transformed cell according to the invention. The transformed cells according to the invention can be used in methods for preparing recombinant polypeptides according to the invention. The methods for preparing a polypeptide according to the invention in recombinant form, characterized in that they use a vector and/or a cell transformed with a vector according to the invention, are themselves included in the present invention. Preferably, a cell transformed with a vector according to the invention is cultured under conditions which allow the expression of said polypeptide, and said recombinant polypeptide is recovered.

As has been mentioned, the cellular host can be chosen from prokaryotic or eukaryotic systems. In particular, it is possible to identify nucleotide sequences according to the invention which facilitate secretion in such a prokaryotic or eukaryotic system. A vector according to the invention carrying such a sequence can therefore be advantageously used for producing recombinant proteins intended to be secreted. As a result, the purification of these recombinant proteins of interest will be facilitated by the fact that they are present in the cell culture supernatant rather than inside the host cells.

The polypeptides according to the invention may also be prepared by chemical synthesis. Such a method of preparation is also a subject of the invention. Those skilled in the art are aware of the methods of chemical synthesis, for example the techniques using solid phases (see in particular Steward et al., 1984, Solid phase peptides synthesis, Pierce Chem. Company, Rockford, 111, 2nd ed., (1984)) or techniques using partial solid phases, by fragment condensation or by conventional synthesis in solution. The polypeptides obtained by chemical synthesis, and possibly comprising corresponding unnatural amino acids, are also included in the invention.

The hybrid polypeptides according to the invention are very useful for obtaining monoclonal or polyclonal antibodies capable of specifically recognizing the polypeptides according to the invention.

These specific polyclonal or monoclonal antibodies can be obtained by the standard methods well known to those skilled in the art, after immunizing a mammal using these polypeptides (or their corresponding nucleic acid) or, for example, according to the conventional method of hybridoma culture described by Köhler and Milstein (1975, Nature, 256, 495) for the monoclonal antibodies.

Such monoclonal or polyclonal antibodies, their fragments, or the chimeric antibodies, which recognize the polypeptides according to the invention, are also subjects of the invention.

The antibodies according to the invention are, for example, chimeric antibodies, humanized antibodies, or Fab or F(ab′)² fragments. They can also be in the form of immunoconjugates or of antibodies which are labeled in order to obtain a detectable and/or quantifiable signal.

Thus, the antibodies according to the invention can be used in a method for detecting and/or identifying bacteria belonging to the genus Photorhabdus and/or to the species Photorhabdus luminescens , in a biological sample, characterized in that it comprises the following steps:
a) bringing the biological sample into contact with an antibody according to the invention;
b) demonstrating the antigen-antibody complex possibly formed.

The antibodies according to the invention can also be used in order to detect expression of a gene of Photorhabdus luminescens TT01. Specifically, the presence of the expression product of a gene recognized by an antibody specific for said expression product can be detected by the presence of an antigen-antibody complex formed after the Photorhabdus luminescens strain TT01 has been brought into contact with an antibody according to the invention. The bacterial strain used may have been “prepared”, i.e. centrifuged, lyzed and/or placed in an appropriate reagent for constituting the medium suitable for the immunoreaction. In particular, preference is given to a method for detecting expression in the gene, corresponding to Western blotting, which may be performed after polyacrylamide gel electrophoresis of a lysate of the bacterial strain, in the presence or absence of reducing conditions (SDS-PAGE). After migration and separation of the proteins on the polyacrylamide gel, said proteins are transferred onto a suitable membrane (for example made of nylon) and the presence of the protein or of the polypeptide of interest is detected by bringing said membrane into contact with an antibody according to the invention.

Thus, the present invention also comprises the kits or sets for carrying out a method as described (for detecting the expression of a gene of Photorhabdus luminescens TT01, or for detecting and/or identifying bacteria belonging to the species Photorhabdus luminescens), comprising the following elements:
a) a polyclonal or monoclonal antibody according to the invention;
b) optionally, the reagents for constituting the medium suitable for the immunoreaction;
c) optionally, the reagents for demonstrating the antigen-antibody complexes produced by the immunoreaction.

The polypeptides and the antibodies according to the invention can advantageously be immobilized on a support, in particular a protein chip. Such a protein chip is the subject of the invention and may also contain at least one polypeptide of a microorganism other than Photorhabdus luminescens , or an antibody directed against a compound of a microorganism other than Photorhabdus luminescens .

The protein chips or high density filters containing proteins according to the invention can be constructed in the same way as the DNA chips according to the invention. In practice, it is possible to carry out the synthesis of the polypeptides directly attached to the protein chip, or to carry out an ex situ synthesis followed by a step of attaching the synthesized polypeptide to said chip. The latter method is preferable when the intention is to attach proteins of considerable size to the support, these proteins being advantageously prepared by genetic engineering. However, if the intention is to attach only peptides to the support of said chip, it may be more advantageous to synthesize said peptides directly in situ.

The protein chips according to the invention can advantageously be used in kits or sets for detecting and/or identifying bacteria related to the species Photorhabdus luminescens , or to a microorganism, or more generally in kits or sets for detecting and/or identifying microorganisms. When the polypeptides according to the invention are attached to DNA chips, the presence of antibodies in the samples tested is sought, the attachment of an antibody according to the invention to the support of said protein chip allowing identification of the protein for which said antibody is specific.

Preferably, an antibody according to the invention is attached to the support of the protein chip, and the presence of the corresponding antigen, specific for Photorhabdus luminescens , is detected.

A protein chip described above can be used to detect gene products, in order to establish an expression profile for said genes, in addition to a DNA chip according to the invention. The protein chips according to the invention are also extremely useful for proteomic experiments which study interactions between the various proteins of a cell of a plant, of an animal, such as an insect, or of a microorganism other than P. luminescens.

Thus, the invention also comprises a protein chip according to the invention, characterized in that it also contains at least one polypeptide of a cell of a plant, of an animal or of a microorganism other than P. luminescens, immobilized on the support of said chip, preferably said cell or other microorganism is chosen from a cell or microorganism sensitive to a toxin or an antibiotic produced by P. luminescens.

In a simplified manner, representative peptides of the various proteins of an organism are attached to a support. Said support is then brought into contact with labeled proteins and, after an optional rinsing step, interactions between said labeled proteins and the peptides attached to the protein chip are detected.

Thus, the protein chips comprising a polypeptide sequence according to the invention or an antibody according to the invention are a subject of the invention, as are the kits or sets containing them.

The present invention also covers a method for detecting and/or identifying bacteria belonging to the species Photorhabdus luminescens , in a biological sample, which uses a nucleotide sequence according to the invention.

It should be understood that, in the present invention, the term “biological sample” concerns samples taken from a living organism (in particular blood, tissues, organs or others taken from a mammal) or a sample containing biological material, i.e. DNA or RNA. Such a biological sample also comprises food compositions containing bacteria (for example cheeses, dairy products), but also food compositions containing yeasts (beers, breads) or others. The term “biological sample” also concerns bacteria isolated from these samples or food compositions.

The method of detection and/or identification using the nucleotide sequences according to the invention may be diverse in nature.

A method comprising the following steps is preferred:
a) optionally isolating the DNA from the biological sample to be analyzed, or obtaining a cDNA from the RNA of the biological sample;
b) specifically amplifying the DNA of bacteria belonging to the species Photorhabdus luminescens using at least one primer according to the invention;
c) demonstrating the amplification products.

This method is based on the specific amplification of the DNA, in particular via a chain amplification reaction.

A method comprising the following steps is also preferred:
a) bringing a nucleotide probe according to the invention into contact with a biological sample, the nucleic acid contained in the biological sample having, where appropriate, previously been made accessible to hybridization, under conditions which allow hybridization of the probe to the nucleic acid of a bacterium belonging to the species Photorhabdus luminescens ;
b) demonstrating the hybrid possibly formed between the nucleotide probe and the DNA of the biological sample.

Such a method should not be limited to detecting the presence of the DNA contained in the biological sample to be tested, it can also be used to detect the RNA contained in said sample. This method encompasses in particular Southern and Northern blotting.

Another preferred method according to the invention comprises the following steps:
a) bringing a nucleotide probe immobilized on a support according to the invention into contact with a biological sample, the nucleic acid of the sample having, where appropriate, previously been made accessible to hybridization, under conditions which allow hybridization of the probe to the nucleic acid of a bacterium belonging to the species Photorhabdus luminescens ;
b) bringing the hybrid formed between the nucleotide probe immobilized on a support and the nucleic acid contained in the biological sample, where appropriate after removing the DNA of the biological sample which has not hybridized with the probe, into contact with a labeled nucleotide probe according to the invention;
c) demonstrating the new hybrid formed in step b).

This method is advantageously used with a DNA chip according to the invention, the nucleic acid being sought hybridizing with a probe present at the surface of said chip, and being detected using a labeled probe. This method is advantageously carried out by combining a prior step of amplifying the DNA or the complementary DNA optionally obtained by reverse transcription, using primers according to the invention.

Thus, the present invention also encompasses the kits or sets for detecting and/or identifying bacteria belonging to the species Photorhabdus luminescens , characterized in that it comprises the following elements:
a) a nucleotide probe according to the invention;
b) optionally, the reagents required for carrying out a hybridization reaction;
c) optionally, at least one primer according to the invention and also the reagents required for a DNA amplification reaction.

Similarly, the present invention also encompasses the kits or sets for detecting and/or identifying bacteria belonging to the species Photorhabdus luminescens TT01, characterized in that it comprises the following elements:
a) a nucleotide probe, termed capture probe, according to the invention;
b) an oligonucleotide probe, termed detection probe, according to the invention;
c) optionally, at least one primer according to the invention and also the reagents required for a DNA amplification reaction.

Finally, the kits or sets for detecting and/or identifying bacteria belonging to the species Photorhabdus luminescens , characterized in that they comprise the following elements:
a) at least one primer or one probe according to the invention;
b) optionally, the reagents required to carry out a DNA amplification reaction;
c) optionally, a component for verifying the sequence of the amplified fragment, more particularly an oligonucleotide probe according to the invention, are also subjects of the present invention.

Preferably, said primers and/or probes and/or polypeptides and/or antibodies according to the present invention, used in the methods and/or kits or sets according to the present invention, are chosen from the primers and/or probes and/or polypeptides and/or antibodies specific for the species Photorhabdus luminescens. Preferably, these elements are chosen from the nucleotide sequences encoding a secreted protein, from the secreted polypeptides, or from the antibodies directed against secreted polypeptides of Photorhabdus luminescens.

A subject of the present invention is also the strains of Photorhabdus luminescens TT01 containing one or more mutation(s) in a nucleotide sequence according to the invention, in particular an ORF sequence, or regulatory elements thereof (in particular promoters).

According to the invention, preference is given to the strains of Photorhabdus luminescens TT01 exhibiting one or more mutation(s) in the nucleotide sequences encoding polypeptides preferably with activity of the toxin or antibiotic type, or involved in their biosynthesis, or else, in another aspect, involved in the cellular machinery, in particular secretion, central intermediate metabolism, energy metabolism, and processes of amino acid synthesis, of transcription and of translation, and of polypeptide synthesis.

Said mutations may lead to inactivation of the gene or, in particular, when they are located in the regulatory elements of said gene, to overexpression of this gene.

According to the present invention, the strains of Photorhabdus luminescens TT01 exhibiting one or more mutation(s) may be used to validate the function of a wild-type gene of Photorhabdus luminescens .

The invention also relates to the use of a nucleotide sequence according to the invention, of a polypeptide according to the invention, of an antibody according to the invention, and/or of a cell according to the invention, for selecting an organic or inorganic compound capable of modulating, regulating, inducing or inhibiting gene expression in a plant or animal cell or in a microorganism other than P. luminescens, the resistance or sensitivity of which to at least one toxin or antibiotic produced by P. luminescens it is, for example, desired to modify.

The invention also comprises a method for selecting compounds capable of binding to a polypeptide or one of its fragments according to the invention, capable of binding to a nucleotide sequence according to the invention, or capable of recognizing an antibody according to the invention, and/or capable of modulating, regulating, inducing or inhibiting gene expression, and/or of modifying growth or cell replication of eukaryotic or prokaryotic cells, or capable of inducing, inhibiting or increasing, in an animal or plant organism, resistance or sensitivity to at least one toxin or antibiotic produced by P. luminescens, said method comprising the following steps:
a) bringing said compound into contact with said polypeptide or said nucleotide sequence and/or with a transformed cell according to the invention;
b) determining the ability of said compound to bind to said polypeptide or said nucleotide sequence, or to modulate, regulate, induce or inhibit gene expression, or to modulate growth or cell replication, or to induce, inhibit or increase, in an animal or plant organism, resistance or sensitivity to at least one toxin or antibiotic produced by P. luminescens.

The transformed cells according to the invention may advantageously be used as a model and may be used in methods for studying, identifying and/or selecting compounds liable to be responsible for resistance or sensitivity to at least one toxin or antibiotic produced by P. luminescens. The compounds which may be selected can be organic compounds such as polypeptides or carbohydrates or any other organic or inorganic compounds which are already known, or new organic compounds developed using molecular modeling techniques and obtained by chemical or biochemical synthesis, these techniques being known to those skilled in the art.

The invention relates to the compounds which can be selected using a method of selection according to the invention.

The invention also relates to a composition, in particular pesticidal or pharmaceutical composition, comprising a compound chosen from the following compounds:
a) a nucleotide sequence according to the invention;
b) a polypeptide according to the invention;
c) a vector according to the invention;
d) an antibody according to the invention; and
e) a compound which can be selected using a method of selection according to the invention,
   optionally in combination with a pharmaceutically acceptable vehicle.

The invention also relates to a pharmaceutical composition according to the invention, for preventing or treating an infection with a microorganism, such as a bacterium or a fungus, sensitive to at least one toxin or antibiotic produced by P. luminescens.

The invention also relates to a pesticidal composition, in particular against insects, bacteria and/or fungi, according to the invention, for preventing or treating plants infested with animals, such as insects, or with a microorganism, such as a bacterium or a fungus, sensitive to at least one toxin or antibiotic produced by P. luminescens.

The invention also comprises the use of a transformed cell according to the invention, for preparing a toxin or an antibiotic produced by P. luminescens.

The expression “pharmaceutically acceptable vehicle” is intended to denote a compound or a combination of compounds making up a pharmaceutical composition causing no side effects and which makes it possible, for example, to facilitate administration of the active compound, to increase its lifetime and/or its effectiveness in the organism, to increase its solubility in solution or else to improve its conservation. These pharmaceutically acceptable vehicles are well known and will be adjusted by those skilled in the art as a function of the nature and of the method of administration of the active compound chosen.

Preferably, these pharmaceutical compounds will be administered systemically, in particular intravenously, intramuscularly, intradermally or subcutaneously, or orally.

Their methods of administration, dosages and pharmaceutical forms which are optimal can be determined according to the criteria generally taken into account in establishing a treatment suitable for a patient, such as, for example, the age or the body weight of the patient, the seriousness of his or her general condition, the tolerance to the treatment and the side effects observed.

Finally, the invention comprises the use of a composition according to the invention, for preparing a medicinal product intended for the prevention or treatment of an infection with a microorganism, such as a bacterium or a fungus, sensitive to at least one toxin or antibiotic produced by P. luminescens.

Moreover, a subject of the present invention is also a genomic DNA library of a bacterium of the genus Photorhabdus, preferably Photorhabdus luminescens , preferably the strain TT01.

The genomic DNA libraries described in the present invention, in particular the BAC library deposited with the CNCM [French National Collection of Cultures and Microorganisms] on May 12, 2000, under No. I-2478 and which covers the Photorhabdus luminescens TT01 genome.

The invention also relates to a method for identifying at least one nucleotide sequence of P. luminescens not present in the genome of another species of bacterium, in particular a pathogenic bacterium, and/or for identifying at least one nucleotide sequence of a genome of a bacterium, in particular a pathogenic bacterium, of a species other than P. luminescens and not present in the P. luminescens genome, characterized in that it comprises the following steps:
a) the nucleotide sequences of P. luminescens according to the invention, or contained in a genomic library according to the invention, are aligned with the genomic sequence of the other bacterial species, or one of its fragments; and
b) the data obtained with this alignment are processed in order to isolate and identify said sequence(s) only present in one or the other genome.

Such a method, also called “subtractive genomic method”, can be used here to identify a sequence responsible for the pathogenicity of a bacterium, such as a gram-negative bacterium, for which the P. luminescens bacterium, which is not pathogenic, can serve as a model for comparison.

The present invention thus relates to the methods for isolating a polynucleotide of interest present in a strain of Photorhabdus and absent from another strain or species, which use at least one DNA library based, for example, on a plasmid pcDNA2.1 containing the Photorhabdus genome. The method according to the invention for isolating a polynucleotide of interest can comprise the following steps:
a) isolating at least one polynucleotide contained in a clone of the library of DNA of Photorhabdus origin;
b) isolating:
   at least one genomic polynucleotide or cDNA of a bacterium, said bacterium belonging to a strain or species different from the Photorhabdus strain used to construct the DNA library of step a) or, alternatively,
   at least one polynucleotide contained in a clone of a DNA library prepared from the genome of a bacterium belonging to a strain or species different from the Photorhabdus strain used to construct the DNA library of step a), and hybridizing the polynucleotide of step a) to the polynucleotide of step b);
c) selecting the polynucleotides of step a) which have not formed a hybridization complex with the polynucleotides of step b);
d) characterizing the selected polynucleotide.

The polynucleotide of step a) can be prepared by digesting at least one recombinant clone with a suitable restriction enzyme and, optionally, amplifying the polynucleotide insert which results therefrom.

Thus, the method of the invention allows those skilled in the art to carry out comparative genomic studies between a bacterium of the genus Photorhabdus and between, for example, a pathogenic strain or species.

In particular, it is possible to study and determine the regions of polymorphism between said strains.

The present invention also relates to the use of the nucleic acid sequences or of the polypeptides according to the invention:
for preparing biopesticides, in particular entomotoxins, antibiotics, antifungal agents or cytotoxins,
for secreting proteins,
as virulence factors,
for control via quorum-sensing, for identifying targets for human diseases for which Photorhabdus luminescens is a model (in particular the plague or whooping cough), and
for identifying targets against pathogenic Gram-negative bacteria using the subtractive genomic method (such as, for example, by comparison with E. coli or other pathogenic gram-negative bacteria).

The present invention also relates to the use of the polypeptides according to the present invention, for screening compounds capable of modulating the activity of these polypeptides, in particular the polypeptides with enzymatic activity.

Other characteristics and advantages of the invention appear in the following examples:

### EXAMPLES

### Example 1

### Materials and Methods

The strategy for sequencing the genome of Photorhabdus luminescens strain TT01 is based on random (shotgun) sequencing. The first step of this study consisted in cloning the genomic DNA of the Photorhabdus luminescens bacterium into various vectors (plasmids and BACs). Photorhabdus luminescens genomic DNA libraries used.

Three genomic DNA libraries were prepared:
I)—A genomic DNA library in a high copy number bacterial vector (pcDNA2.1, Invitrogen). Average insert size 1.5 kb.
II)—A genomic DNA library in a low copy number bacterial vector (pSYX34). Average insert size 10 kb.
III)—A genomic DNA library in a BAC vector (pBeloBAC11, California Institute of Technology). Average insert size 50 to 100 kb.

Two batches of DNA of the TT01 strain of the Photorhabdus luminescens bacterium were extracted on Oct. 30, 1998 and Apr. 14, 1999.

I) Establishing a Genomic DNA Library in the Bacterial Vector pcDNA2.1 (Invitrogen)

A) Preparation of the Vector: pcDNA2.1

We prepared the plasmid pcDNA2.1 by carrying out two midipreps (QIAGEN KIT) in parallel according to the conditions recommended by the manufacturer. The vector pcDNA2.1 was digested with the BstX1 restriction enzyme.

B) Preparation of the Chromosomal DNA of Photorhabdus luminescens Strain TT01 and Ligation into the Vector pcDNA2.1

1) Dissolving of the DNA

A dry pellet of genomic DNA of the strain TT01, prepared on Oct. 30, 1998, was taken up in 200 μl of 10:1 TE, and then dissolved for 30 minutes at 65° C. Its concentration was estimated at 0.15 μg/μl.

2) Nebulization of the DNA

50 μl of genomic DNA strain TT01 in an amount of H₂O sufficient for 2 ml were nebulized for 45 sec at a pressure of 1 bar of nitrogen, and centrifuged for 2 min at 600 rpm in order to recover the entire volume.

3) Precipitation of the DNA

This DNA was then precipitated with sodium acetate (2 ml of DNA+0.2 ml of 3M Na acetate, pH 5.2,+5 ml of absolute ethanol; 2 h at −20° C.), centrifuged for 30 min at 14 000 rpm and at 4° C., and then redissolved in 100 μl of water.

4) Analysis of the DNA

4 μl were loaded onto a 1% agarose TBE gel.

DNA fragments of the expected size of 500 bp to 3 kb were visualized.

5) Repair of the DNA

In order to blunt end the DNA fragments, repair was carried out using T4 DNA polymerase.

The following are mixed in 2 separate tubes:
48 μl of DNA from step 3),
100 μl of H₂O,
20 μl of 5× ligation buffer,
2 μl of dNTP mix (10 mM),
5 μl of T4 DNA polymerase (Boehringer).

Incubation is carried out for 25 min at ambient temperature and the reaction is then stopped by heating for 15 min at 75° C.

6) Precipitation of the DNA

This DNA was then precipitated overnight at −20° C. with sodium acetate (1/10 volume of Na acetate and 2.5 volume of absolute ethanol) and centrifuged, and the DNA pellet obtained was then air-dried and redissolved in 30 μl of water.

7) Ligation of the DNA

This DNA was ligated overnight at 16° C. with the Bstx A+Bstx B linkers (Invitrogen) in the presence of 2 μl of ligase.

8) Preparation of the Inserts

After migration of the DNA ligated to the linkers in a 1% agarose TAE gel, at 70 volts, the region of interest (between 1 and 3 Kb) was cut into four fragments.

9) Purification of the Inserts

The agarose fragments containing the regions of interest were purified using Geneclean (BIO101) according to the conditions recommended by the manufacturer.

An aliquot was loaded onto an agarose minigel in order to validate the quality of the purification.

10) Ligation of the Inserts into the Vector pcDNA2.1 (Invitrogen)
4 μl of DNA (insert after the Geneclean purification step, step 9),
2 μl of plasmid pcDNA2.1 (after the BstX1 digestion step and then Geneclean purification),
2 μl of ligation buffer (10×),
add 2 μl of ligase,
10 μl H₂O (total volume: 20 μl).

The mixture is incubated overnight at 16° C.

11) Transformation of Ultracompetent XL2 Blue Cells (Stratagene)

The genomic DNA library obtained in step 10 was integrated into ultracompetent XL2 Blue cells (Stratagene) according to the conditions recommended by the manufacturer.

Analysis of the inserts of 24 clones by digestion with the PvuII restriction enzyme and by sequencing of the ends was carried out and gave satisfactory results.

II) Establishing a Genomic DNA Library in the Bacterial Vector pSYX34

The PARTIAL FILL-IN technique, developed in the laboratory, allowed us to construct a library of genomic DNA of the TT01 strain of the bacterium Photorhabdus luminescens , cloned into the plasmid pSYX34 (average insert size 10 Kb).

Note: in this case, digestion of the vector pSYX34 with the Sal I restriction enzyme frees the following ends:

5′TCGAC-G-5′

The PARTIAL FILL-IN was carried out in the presence of dCTP and dTTP deoxynucleotides.

5′TCGAC-CTG-5′

A) Preparation of the Vector: pSYX34

1) Production of the Vector

Plasmid pSYX34 was prepared by carrying out two midipreps, QIAGEN KIT, in parallel according to the conditions recommended by the manufacturer.

2) Digestion of the Plasmid pSYX34 with the Sal I Restriction Enzyme

The final volume will be 100 μl:
20 μl pSYX34,
10 μl of buffer H,
66 μl of H₂O,
4 μl Sal I.

The mixture is incubated for 2 h at 37° C.

An aliquot and also a molecular weight marker were loaded onto an agarose minigel in order to validate the quality of the purification.

3) Chloroform Extraction

Chloroform extraction makes it possible to stop the enzyme digestion and to remove any traces of protein.

After digestion, 95 μl are recovered,

105 μl of 10 mM TE are added,

200 μl of chloroform are added.

The mixture is vortexed and centrifuged for 1 min at 1 000 rpm.

The aqueous phase (upper phase) is recovered.

4) Precipitation with Sodium Acetate
1/10 volume of sodium acetate (3M, pH: 5.2), i.e. 20 μl, are added,
followed by 2.5 volumes of clean absolute ethanol, i.e. 500 μl (bottle kept at −20° C.).
The mixture is left at −20° C. for at least 1 hour (it is possible to leave it at −20° C. overnight).
The mixture is centrifuged at 4° C. (cold room) for 30 min at 14 000 rpm.
The supernatant is removed using the vacuum pump.
400 μl of 70% ethanol are added and the mixture is centrifuged at 4° C. for 5 min at 14 000 rpm.
The supernatant is removed using the vacuum pump and the pellet is left to dry for approximately one hour on the bench.
The pellet is resuspended in 20 μl of 10 mM TE (1/10).

5) Partial Fill-in

Final volume of the reaction: 50 μl; 20 μl of pSYX34 digested with Sal I
5 μl of synthesis buffer,
2.5 μl of 1 mM nucleotide (C-T) mix,
20.5 μl of water,
2 μl of Klenow (2 U/μl).

The mixture is left for 30 min at ambient temperature.

Verification is carried out on a 1% agarose minigel, followed by freezing at −20° C.

Mixture of C-T Nucleotides:

Mix:
2 μl of T nucleotides (100 mM),
2 μl of C nucleotides (100 mM),
16 μl of 10 mM TRIS, pH=7.5.

The nucleotides are thus diluted to 1/10, for a concentration of 10 mM.

A second dilution to 1/10 in 10 mM TRIS buffer will be carried out so as to have a concentration of 1 mM.

In addition, during the reaction, the nucleotides are diluted to 1/20 (2.5 μl in 50 μl), thus obtaining a final concentration of nucleotides of 50 μM.

6) Purification of the Vector pSYX34: Preparative Gel

Prepare a 1% agarose TAE gel, large wells will be provided for the samples.

Take 30 μl of pGB2 after the partial fill-in (20 μl will therefore remain in the freezer), add 6 μl of loading solution. In parallel, load the molecular weight marker in the following proportions:
5 μl H₂O,
5 μl of the 1 Kb DNA marker,
2 μl of the loading solution.

Allow to migrate at 70 volts.

Cut the gel on the marker side using a scalpel and locate, by taking a photo and using a ruler, the region of interest; in our case, the band corresponding to 4 Kb will be taken. Each sample will be cut in half. The weight of each sample will indicate its approximate volume.

There will therefore be four samples to purify by the Geneclean II technique.

7) Purification of the Vector pSYX34
Take up in 3 volumes of sodium iodide solution (1 ml for a sample of 0.2 g), place at 49° C., shaking every 2 minutes, until the agarose has completely dissolved.
Add 8 μl of microbeads: GLASSMILK R, leave at ambient temperature for 5-10 min, the DNA attaches to the microbeads.
Centrifuge for 2 min at 14 000 rpm, remove the supernatant by suction.
Wash 3 times with the New Wash solution (600 μl/eppendorf, vortex, centrifuge for a few seconds at 10 000 rpm); this solution was prepared and is kept at −20° C.
Resuspend the pellet in 10 μl of H₂O, vortex, leave for 5 min at 49° C. This allows the DNA to detach from the microbeads.
Centrifuge for 2 min at 14 000 rpm.
Recover the supernatants in new eppendorfs (No. 1 and No. 2, etc)
Add 10 μl of H₂O again to the pellets, so as to be sure to recover everything, vortex, leave for 2 min at 49° C.
Centrifuge for 2 min at 14 000 rpm.
Recover the supernatants in the preceding eppendorfs (No. 1 and No. 2, etc).
There are approximately 20 μl of supernatant, centrifuge for 2 min at 14 000 rpm.
Transfer the supernatants into new eppendorfs, so as to be sure that there are no longer any microbeads.
Load 1 μl of each preparation (+5 μl of H₂O+2 μL of loading solution) onto a 1% agarose minigel, allow to migrate at 80 volts for 2 hours.

The most concentrated samples will be kept, they will then be frozen at −20° C.

B) Preparation of the Chromosomal DNA of Photorhabdus luminescens Strain TT01

1) Dissolving of the DNA

A dry pellet of genomic DNA of the strain TT01 was taken up in 200 μl of 10:1 TE and then dissolved for 30 min at 65° C. Its concentration was estimated at 0.15 μg/μl.

2) Partial Digestion of the Genomic DNA with the Sau3A Restriction Enzyme

15 μl of DNA were digested with 2, 1, 0.5, 0.25, 0.125, 0.0625 or 0.03125 units of Sau3A restriction enzyme for 1 h at 37° C.

3) Chloroform Extraction

Chloroform extraction makes it possible to stop the enzyme digestion and to remove any traces of protein.

After digestion, 100 μl of chromosomal DNA are recovered.

100 μl of 10 mM TE are added,

200 μl of chloroform are added.

The mixture is vortexed and centrifuged for 1 min at 1 000 rpm.

The aqueous phase, the upper phase, comprising the chromosomal DNA is recovered.

4) Precipitation with Sodium Acetate

1/10 volume of sodium acetate (3M, pH: 5.2), i.e. 20 μl, are added.

2.5 volumes of clean absolute ethanol, i.e. 500 μl, (bottle kept at −20° C.) are then added.

The mixture is left at −20° C. for at least 1 hour (it is possible to leave it at −20° C. overnight).

The mixture is centrifuged at 4° C. (cold room) for 30 min at 14 000 rpm.

The supernatant is removed using the vacuum pump.

400 μl of 70% ethanol are added, the mixture is centrifuged at 4° C. for 5 min at 14 000 rpm.

The supernatant is removed using the vacuum pump and the pellet is left to dry for approximately one hour on the bench.

The pellet is resuspended in 20 μl of H₂O.

5) Verification of the Partial Digestions, after Precipitation with Sodium Acetate.

A 1% agarose TBE gel is prepared. 1/10 of the total volume of the precipitations, i.e. 2 μl of DNA+8 μl of H₂O+2 μl of loading solution, is loaded.

In parallel, a molecular weight marker is loaded: 1 μl+9 μl of H₂O+2 μl of loading solution.

6) Partial Fill-in

Final volume of the reaction: approximately 50 μl
36 μl of partially digested DNA (DNA digested with 0.25, 0.125, 0.0625 or 0.03125 units of Sau3A restriction enzyme)
5 μl of synthesis buffer
10 μl of 1 mM nucleotide (A-G) mix=>final concentration: 200 μM
2 μl of Klenow (2 U/μl).

The mixture is left at ambient temperature for 30 min.

Mixture of A-G Nucleotides

Mix:
2 μl of A nucleotides (100 mM)
2 μl of G nucleotides (100 mM)
16 μl of 10 mM TRIS, pH=7.5.

The nucleotides are thus diluted to 1/10, giving a concentration of 10 mM.

A second dilution to 1/10 in H₂O will be carried out, a concentration of 1 mM is therefore obtained.

7) Chloroform Extraction

Chloroform extraction makes it possible to stop the enzyme digestion and to remove any traces of protein.

After digestion, 53 μl of chromosomal DNA are recovered.

47 μl of 10 mM TE are added.

100 μl of chloroform are added.

The mixture is vortexed and centrifuged for 1 min at 1 000 rpm.

The aqueous phase, the upper phase, comprising the chromosomal DNA is recovered.

8) Precipitation with Sodium Acetate

1/10 volume of sodium acetate (3M, pH: 5.2), i.e. 10 μl, are added.

2.5 volumes of clean absolute ethanol, i.e. 250 μl, (bottle kept at −20° C.) are then added.

The mixture is left at −20° C. for at least 1 hour (it is possible to leave it at −20° C. overnight).

The mixture is centrifuged at 4° C. (cold room) for 30 min at 14 000 rpm.

The supernatant is removed using the vacuum pump.

400 μl of 70% ethanol are added, the mixture is centrifuged at 4° C. for 5 min at 14 000 rpm.

The supernatant is removed using the vacuum pump and the pellet is left to dry for approximately one hour on the bench.

The pellet is resuspended in 20 μl of H₂O.

9) Purification of the Chromosomal DNA: Preparative Gel

Prepare a 1% agarose TAE gel, large wells will be provided for the samples.

Take the entire DNA, i.e. 20 μl, add 4 μl of loading solution. In parallel, load the molecular weight marker in the following proportions:
5 μl H₂O
5 μl of the 1 Kb DNA marker
2 μl of the loading solution.

Allow to migrate at 70 volts.

Cut the gel on the marker side using a scalpel and locate, by taking a photo and using a ruler, the region of interest. Since the DNA fragments are long, SPIN X columns from COSTAR will be used to purify the DNA, this will avoid cleaving the chromosomal DNA.

10) Purification: SPIN X Column

After having recovered the various pieces of agarose, in SPIN X columns, add 200 μl of 10 mM TE and then grind using a spatula.

This may be kept at 4° C. Then, centrifuge for 20 min at 5 000 rpm. The agarose will be retained on the filters, whereas the chromosomal DNA will be found at the bottom of the tube.

11) Chloroform Extraction

Chloroform extraction makes it possible to remove any traces of protein.

800 μl of chromosomal DNA are recovered.

800 μl of chloroform are added.

The mixture is vortexed and centrifuged for 1 min at 1 000 rpm.

The aqueous phase, the upper phase, comprising the chromosomal DNA is recovered.

12) Precipitation with Sodium Acetate

1/10 volume of sodium acetate (3M, ph: 5.2), i.e. 80 μl, are added.

800 μl of isopropanol are then added.

The mixture is left at −20° C. for at least 1 hour (it is possible to leave it at −20° C. overnight).

The mixture is centrifuged at 4° C. (cold room) for 30 min at 14 000 rpm.

The supernatant is removed using the vacuum pump.

400 μl of 70% ethanol are added and the mixture is centrifuged at 4° C. for 5 min at 14 000 rpm.

The supernatant is removed using the vacuum pump, and the pellet is allowed to dry for approximately half an hour on the bench and 2 min under vacuum.

The pellet is resuspended in 20 μl of TE (0.1×, i.e. 1 mM).

The suspension is incubated for 10 min at 65° C.

The suspension is incubated for approximately 4 hours at 4° C., so that the DNA is well resuspended.

Verification is carried out on a 1% agarose minigel with a molecular weight marker placed in parallel.

C) Ligation of the chromosomal DNA of Photorhabdus luminescens Strain TT01 into the Vector pSYX34

Take 6 μl of chromosomal DNA.

Add 2 μl of pSYX34.

Add 2 μl of ligation buffer (LB).

Add 2 μl of ligase.

Then add 8 μl of H₂O (total volume: 20 μl)

In parallel, prepare a ligation control by replacing the chromosomal DNA with 0.1× TE (1 mM).

Vortex, leave overnight at 16° C.

Transformation of Ultracompetent XL10-Gold Kanr Cells (Stratagene)

The genomic DNA library obtained in the preceding step was integrated into ultracompetent XL10 Gold Kanr cells (Stratagene) according to the conditions recommended by the manufacturer.

Analysis of the inserts of 24 clones by digesting with the SalI restriction enzyme and by sequencing of the ends was carried out and gave satisfactory results.

III) Establishing a Genomic DNA Library in the Bacterial Vector pBeloBAC11 (California Institute of Technology)

Construction of the BAC Library

Partial digestion of the DNA in pieces of agarose:
the pieces of agarose are washed three times for 15 minutes at ambient temperature in a solution of TE (1×) with moderate agitation;
the pieces of agarose are equilibrated twice in 300 μl of a Hind III digestion buffer (1×) (Boehringer or Biolabs) for 30 minutes at ambient temperature;
the digestion buffer is removed and an ice-cold Hind III digestion buffer (1 ml per piece of agarose) containing 20 U of Hind III (Boehringer or Biolabs) is added;
incubation is carried out for two hours in ice;
the pieces of agarose are transferred to a water bath at 37° C. and incubation is carried out for 10 minutes to 30 minutes (depending on the DNA contained in the pieces of agarose); and
the digestion is stopped by adding 100 μl of a 250 mM EDTA solution (pH 8).

Size Selection:

Separation of the Partially Digested DNA by PFGE Using the CHEF DRIII Apparatus (Bio-Rad):
gel of 1% LMP agarose in a tris-acetate-EDTA buffer (1×) at 13° C.,
reverse the current every 3 to 15 seconds at 6 V/cm for 16 hours,
load at least two pieces of agarose and the marker,
stain the marker and one lane or a part of the lane to verify the partial digestion,
excise the bands (unstained part) of various sizes (i.e. from 50 to 100 kb and from 150 to 250 kb),
the agarose bands can be stored at 4° C. in a TE solution.

Preparation of the Vector:
isolation of pBeloBACII by the cesium chloride method;
digestion with Hind III;
dephosphorylation with CIP (optionally with HK phosphatase).

Ligation and Transformation:
the agarose banks containing the DNA are melted at 60° C. for 10 minutes;
the molten solution of agarose/DNA is equilibrated for 15 minutes at 45° C.;
gelase (Epicentre Technologies) is added in a proportion of 1 U per 100 μl of gel band (do not add digestion buffer, which causes certain problems at the time of ligation);
the digestion is carried out for 1 hour at 45° C.;
the ligation is carried out in 50 μl of a solution containing pBeloBACII (2 μl), T4 ligase (1 μl at 1:10), a 10× T4 buffer (5 μl ), and the DNA/agarose solution (42 μl), incubation is for 20 hours at 16° C.;
the ligation medium is heated for 15 minutes at 65° C.;
the ligation medium is dialyzed against a Tris-EDTA buffer, using Millipore membranes of pore size VS 0.025 mM;
1 or 2 μl of the ligation solution are introduced by electroporation into E. coli DH10B cells (Gibco BRL) using electroporation cuvettes with a width of 2 mm, with the following settings: 2.5 kV, 25 μF and 200Ω;
after electroporation, the cells are resuspended in 600 μl of SOC or NYZ medium, and then incubation is carried out for 45 minutes at 37° C. with agitation;
10 and 100 μl of each cell suspension are plated out in an LB agar medium containing chloramphenicol (12.5 μg/ml), X-Gal (5-bromo-4-chloro-3-indolyl-α-D-glucuronic acid; 50 μg/ml) and IPTG (isopropyl-α-D-thiogalactopyranoside; 25 μg/ml); and
the dishes thus obtained are incubated overnight.

Extraction of the Plasmid DNA and Sequencing of the Inserts

The plasmid DNA was extracted by the alkaline lysis technique in 24-well plates.

The inserts of the clones were sequenced using the PE Big Dye kit according to the conditions recommended by the manufacturer, using the oligonucleotides specific for each vector.

The reactions are then introduced into the thermocycler in order to undergo 35 cycles composed of the following three steps:
denaturation for 10 seconds at 96° C.,
hybridization for 10 seconds at 50° C.,
elongation for 4 minutes at 56° C.

A precipitation with 76% ethanol is then carried out for 20 minutes at ambient temperature.

The plates are then centrifuged for 35 minutes at 2 200 g, then drained on absorbent paper, and then centrifuged turned upside down on absorbent paper for 1 minute at 500 g in order to leave no trace of ethanol.

The DNA pellets are then:
either taken up in a solution of formamide-EDTA-dextran blue, and then denatured for 2 minutes at 96° C. The plates are then immediately placed in ice, an aliquot then being loaded onto an acrylamide gel of a PE-377 automatic sequencer,
or taken up in a 0.3 mM EDTA solution, an aliquot then being automatically loaded into a PE-3700 automatic sequencer.

Assembly of the Sequences Obtained

The genomic DNA sequences were assembled in a contig using the PHRED-PHRAP software and visualized using the CONSED software.

Analysis and Annotation

The contigs were first of all analyzed and annotated automatically using the GMPTB software.

### Example 2

### The Genes of Interest

The various characteristics associated with the way of life of Photorhabdus luminescens and reported in the literature (Forst et al., 1997; Hu et al., 1998, etc.) led to the presence of genes involved in the biosynthesis of antibiotics and of toxins being sought in the genome of this bacterium.

The Operons for Biosynthesis of Antibiotics

The search for peptide synthetases was undertaken in silico. To do this, we initially used the various motifs conventionally described in the literature (Stachelhaus & Marahiel, 1995, FEMS Microbiology Letters 125:3-14) as a probe in order to locate, by sequence homology, in the Photorhabdus luminescens genome, genes which may be involved in the biosynthesis of antibiotics.

Adenylation Modules

1 - LKAGGAYYVPID
2 - YSGTTGxPKGV
3 - GELCIGGXGxARGYL
4 - YxTGD
5 - VKIRGxRIELGEIE

Thioester Formation Module

6 - DNFYxLGGHSL

N-Methylation Module

VLE/DxGxGxG

Peptide Elongation Module

His-HHILxDGW

Peptide Racemization Module (Optional)

His-HHILxDGW
A - AYxTExNDILLTAxG
B - EGHGRExIIE
C - RTVGWFTSMYPxxLD
D - FNYLGQFD

Among these various modules, some enabled us to identify, by homology search using the BLASTP—basic local alignment search tool program—(Altschul S F et al., 1990, J. Mol. Biol., 215:403-10), genes encoding proteins containing some of these motifs.

The genes thus identified, probably encoding proteins involved in the biosynthesis of antibiotics, were cataloged and appear in particular in table I.

Emphasis was placed particularly on the genes homologous to the syr E gene of Pseudomonas syringae or to the nosD gene.

### REFERENCES

Eric Guenzi, Giuliano Galli, Ingeborg Grgurina, Dennis C. Gross, and Guido Grandi, 1998. Characterization of the Syringomycin Synthetase Gene Cluster. A link between prokaryotic and eukaryotic peptide synthetases. J. Biol. Chem., 273:32857-32863.
Bondi M, Messi P, Sabia C, Baccarani Contri M, Manicardi G., 1999. Antimicrobial properties and morphological characteristics of two Photorhabdus luminescens strains. New Microbiol., 22:117-27.
   The Operons for Biosynthesis of Toxins

As for the genes for biosynthesis of antibiotics, we sought, by sequence homology, in the Photorhabdus luminescens genome, genes potentially encoding toxic proteins. We identified many genes encoding proteins having these criteria.

Entomotoxins (Tc loci).

Homologs of the Tox A gene of Clostridium difficile.

Hemolysins.

Genes potentially encoding proteins homologous to the RTX toxins of Vibrio cholerae.

Genes potentially encoding proteins homologous to the tphA and icmF genes of Legionella pneumophila (Purcell M, Shuman H A, 1998. The Legionella pneumophila icmGCDJBF genes are required for killing of human macrophages. Infect Immun., 66:2245-55).

See annotation of these genes in table I below.

### REFERENCES

Ffrench-Constant R, Bowen D., 1999. Photorhabdus toxins: novel biological insecticides. Curr Opin Microbiol., 2:284-8. Review.
Blackburn M, Golubeva E, Bowen D, Ffrench-Constant R H, 1998. A novel insecticidal toxin from Photorhabdus luminescens, toxins complex a (Tca), and its histopathological effects on the midgut of manduca sexta. Appl Environ Microbiol., 64:3036-41.
Bowen D J, Ensign J C, 1998. Purification and characterization of a high-molecular-weight insecticidal protein complex produced by the entomopathogenic bacterium Photorhabdus luminescens . Appl Environ Microbiol., 64:3029-35.
Bowen D, Rocheleau T A, Blackburn M, Andreev 0, Golubeva E, Bhartia R, Ffrench-Constant R H, 1998. Insecticidal toxins from the bacterium Photorhabdus luminescens. Science., 280:2129-32.
Guo L, Fatig R O 3rd, Orr G L, Schafer B W, Strickland J A, Sukhapinda K, Woodsworth A T, Petell J K, 1999. Photorhabdus luminescens W-14 insecticidal activity consists of at least two similar but distinct proteins. Purification and characterization of toxin A and toxin B. J. Biol Chem., 274:9836-42.

### GENERAL REFERENCES

1. Altschul, S. F., T. L. Madden, A. A. Schaffer, J. Zhang, Z. Zhang, W. Miller, and D. J. Lipman. 1997. Gapped BLAST and PSI-BLAST: a new generation of protein database search programs. [Review] [90 refs]. Nucleic Acids Research. 25:3389-402.
2. Birnboim, H. C. 1983. A rapid alkaline extraction method for the isolation of plasmid DNA. Methods Enzymol. 100:243-255.
3. Braun, L. F. Nato, B. Payrastre, J. C. Mazie, and P. Cossart. 1999. The 213-amino-acid leucine-rich repeat region of the Photorhabdus luminescens InIB protein is sufficient for entry into mammalian cells, stimulation of P1 3-kinase and membrane ruffling. Mol. Microbiol. 34:10-23.
4. Buchrieser, C., C. Rusniok, L. Frangeul, E. Couvé, A. Billault, F. Kunst, E. Carniel, and P. Glaser. 1999. The 102 kb locus of Yersinia pestis: sequence analysis and comparison of selected regions among different Yersinia pestis and Yersinia pseudotuberculosis strains. Infect. Immun. 67:4851-4861.
5. Ewing, B., and P. Green. 1998. Base-calling of automated sequencer traces using phred. II. Error probabilities. Genome Res. 8:186-194.
6. Fitch, W. S. 1970. Distinguishing homologous from analogous proteins. Syst. Zool. 19:99-113.
7. Frangeul, L., K. E. Nelson, C. Buchrieser, A. Danchin, P. Glaser, and K. F. 1999. Cloning and assembly strategies in microbial genome projects. Microbiology. 145:2625-2634.
8. Gordon, D., C. Abajian, and P. Green. 1998. Consed: a graphical tool for sequence finishing. Genome Res. 8:195-202.
9. Jacquet, C., J. Bille, and J. Rocourt. 1992. Typing of Photorhabdus luminescens by restriction polymorphism of the ribonucleic acid gene region. Zentralbl Bakteriol. 276:356-365.
10. Li, P., K. C. Kupfer, C. J. Davies, D. Burbee, G. A. Evans, and H. R. Garner. 1997. PRIMO: A primer design program that applies base quality statistics for automated large-scale DNA sequencing. Genomics. 40:476-485.
11. Lukashin, A. V., and M. Borodovsky. 1998. GeneMark.hmm: new solutions for gene finding. Nucleic Acids Res. 15:1107-1115.
   Scientific Description of the Photorhabdus luminescens Strain TT01 BAC Library Deposited with the CNCM [French National Collection of Cultures and Microorganisms] on May 12, 2000, Under the Number I-2478

Collection of Escherichia coli DH 10B™ clones (Calvin et al., J. Bacteriol. 170, 2796, 1988) containing genomic DNA fragments from the Photorhabdus luminescens strain TT01 bacterium, cloned into the vector pBelo BACII (Kim et al., Genomics, 34, 213, 1996) at the Hind III site. The average insert size is 60 kb.

Databanks

Local revisions of main public banks were used. The protein bank used consists of the nonredundant fusion of these banks, such as the Genpept bank (automatic translation of GenBank and NCBI).

Use was made of the BLAST software package (public domain, Altschul et al., 1990) for searching for homologies between a sequence and protein or nucleic acid databanks. The significance thresholds used depend on the length and on the complexity of the region tested and also on the size of the reference bank. They were adjusted and adapted to each analysis.

The results of the search for homologies between a sequence according to the invention and protein or nucleic acid databanks are given and summarized in table I and table II below.

Table I

List of putative functions (column “homology with nonredundant protein database-description”, and column “annotations” for the sequences selected as being involved in an activity or having an activity of the toxin or antibiotic type) of the proteins of sequences SEQ ID No. 42 to SEQ ID No. 3855 encoded by their respective nucleotide sequence of the Photorhabdus luminescens strain TT01 genome (genome represented by the sequences of the 41 contigs (SEQ ID No. 1 to SEQ ID No. 41)).

The nucleic acid sequences of the proteins of sequence SEQ ID No. 42 to SEQ ID No. 3855 can be easily identified by their start position (“start” column) and end position (“end” column) on each one of the contig sequences (“contig” column).

Legend of Table I:

All of the putative functions associated with the proteins of sequence SEQ ID No. 42 to SEQ ID No. 3855 were obtained through a homology search using in particular BLASTP software (Altschul et al., 1990). The significant identities, or the presence of various motifs (cf. examples) representative of these functions, were in particular taken into account. The description of the most probable function(s) is given in the “homology with” and “Annotation” column.

Table II

List of putative functions (column “function obtained by comparison on databank”) of the proteins encoded by the sequences SEQ ID No. 5835 to SEQ ID No. 10784 of the Photorhabdus luminescens strain TT01 genome (genome here represented by the sequences of the 9 contigs (SEQ ID No. 5826 to SEQ ID No. 5834)).

The nucleic acid sequences of sequence sequences SEQ ID No. 5835 to SEQ ID No. 10784 can be easily identified by their start position (“CONTIG”, “from” column) and end position (“CONTIG” “to” column) on each one of the contig sequences.

Legend of Table II:

All of the putative functions associated with the proteins encoded by the sequences SEQ ID No. 5835 to SEQ ID No. 10784 were obtained through a homology search using in particular BLASTP software (Altschul et al., 1990). The significant identities, or the presence of various motifs (cf. examples) representative of these functions, were in particular taken into account. The description of the most probable function(s) is given in the “homology with” and “Annotation” column.

In the final column, “HOMOLOGOUS TO SEQUENCE SEQ ID”, the term “#N/A” means that no homology for the sequence concerned had been identified with one of the sequences SEQ ID Nos. 42 to 3855. When a homologous sequence was found among the sequences SEQ ID Nos. 42 to 3855 for the sequence concerned, this is mentioned via its SEQ ID number in this column.

LENGTHY TABLE REFERENCED HERE
US20070020625A1-20070125-T00001
Please refer to the end of the specification for access instructions.

LENGTHY TABLE REFERENCED HERE
US20070020625A1-20070125-T00002
Please refer to the end of the specification for access instructions.

LENGTHY TABLE
The patent application contains a lengthy table section. A copy of the table is available in electronic form from the USPTO web site (http://seqdata.uspto.gov/?pageRequest=docDetail&DocID=US20070020625A1) An electronic copy of the table will also be available from the USPTO upon request and payment of the fee set forth in 37 CFR 1.19(b)(3).

1-56. (canceled)

57. An isolated nucleotide sequence derived from the Photorhabdus luminescens genome, comprising a sequence selected from the group consisting of SEQ ID No. 1 to SEQ ID No. 41 and SEQ ID No. 5826 to SEQ ID No. 5834.

58. An isolated nucleotide sequence derived from the Photorhabdus luminescens genome, selected from the group consisting of:
a) a nucleotide sequence comprising at least 75% identity with a sequence chosen selected from the group consisting of SEQ ID No. 1 to SEQ ID No. 41 and SEQ ID No. 5826 to SEQ ID No. 5834;
b) a nucleotide sequence comprising a fragment of a sequence selected from the group consisting of SEQ ID No. 1 to SEQ ID No. 41 and SEQ ID No. 5826 to SEQ ID No. 5834;
c) a nucleotide sequence complementary to a nucleotide sequence as defined in a) or b);
d) a nucleotide sequence of the RNA corresponding to one of the sequences as defined in a), b) or c); and
e) a nucleotide sequence as defined in a), b), c) or d), which has been modified.

59. The nucleotide sequence as claimed in claim 58, wherein the nucleotide sequence is a fragment of a nucleotide sequence selected from the group consisting of SEQ ID No. 1 to SEQ ID No. 41 and SEQ ID No. 5826 to SEQ ID No. 5834, and wherein said fragment encodes a polypeptide selected from the group consisting of SEQ ID No. 42 to SEQ ID No. 3855 or a polypeptide encoded by a sequence selected from the group consisting of SEQ ID No. 5835 to SEQ ID No. 10784.

60. The nucleotide sequence as claimed in claim 59, wherein:
a) said nucleotide sequence encodes a polypeptide selected from the group consisting
of SEQ ID No. 61, SEQ ID No. 62, SEQ ID No. 67, SEQ ID No. 171, SEQ ID No. 221, SEQ ID No. 268, SEQ ID No. 288, SEQ ID No. 380, SEQ ID No. 426, SEQ ID No. 438, SEQ ID No. 448, SEQ ID No. 453, SEQ ID No. 455, SEQ ID No. 456, SEQ ID No. 458, SEQ ID No. 501, SEQ ID No. 516, SEQ ID No. 530, SEQ ID No. 542, SEQ ID No. 551, SEQ ID No. 720, SEQ ID No. 761, SEQ ID No. 762, SEQ ID No. 814, SEQ ID No. 859, SEQ ID No. 860, SEQ ID No. 861, SEQ ID No. 862, SEQ ID No. 869, SEQ ID No. 1079, SEQ ID No. 1168, SEQ ID No. 1174, SEQ ID No. 1176, SEQ ID No. 1413, SEQ ID No. 1414, SEQ ID No. 1415, SEQ ID No. 1416, SEQ ID No. 1417, SEQ ID No. 1457, SEQ ID No. 1651, SEQ ID No. 1856, SEQ ID No. 1869, SEQ ID No. 2021, SEQ ID No. 2080, SEQ ID No. 2152, SEQ ID No. 2162, SEQ ID No. 2173, SEQ ID No. 2251, SEQ ID No. 2295, SEQ ID No. 2306, SEQ ID No. 2317, SEQ ID No. 2328, SEQ ID No. 2340, SEQ ID No. 2342, SEQ ID No. 2351, SEQ ID No. 2500, SEQ ID No. 3228, SEQ ID No. 3230, SEQ ID No. 3311, SEQ ID No. 3317, SEQ ID No. 3318, SEQ ID No. 3319, SEQ ID No. 3320, SEQ ID No. 3322, SEQ ID No. 3323, SEQ ID No. 3326, SEQ ID No. 3327, SEQ ID No. 3328, SEQ ID No. 3375, SEQ ID No. 3376, SEQ ID No. 3377, SEQ ID No. 3378, SEQ ID No. 3422, SEQ ID No. 3489, SEQ ID No. 3503, SEQ ID No. 3609, SEQ ID No. 3623, SEQ ID No. 3624, SEQ ID No. 3772, SEQ ID No. 3783, SEQ ID No. 3788 and SEQ ID No. 3794; or
b) said nucleotide sequence is selected from the group consisting of SEQ ID No. 5835 to SEQ ID No. 10784.

61. A nucleotide sequence, comprising a nucleotide sequence selected from the group consisting of:
a) a nucleotide sequence as claimed in claim 59;
b) a nucleotide sequence comprising at least 75% identity with a nucleotide sequence as claimed in claim 59;
c) a complementary or RNA nucleotide sequence corresponding to a sequence as defined in a) or b);
d) a nucleotide sequence of a representative fragment of a sequence as defined in a) or c); and
e) a sequence as defined in a) or c), which has been modified.

62. A polypeptide encoded by a nucleotide sequence as claimed in claim 58.

63. A polypeptide as claimed in claim 62, wherein:
a) said polypeptide is selected from the group consisting of
SEQ ID No. 61, SEQ ID No. 62, SEQ ID No. 67, SEQ ID No. 171, SEQ ID No. 221, SEQ ID No. 268, SEQ ID No. 288, SEQ ID No. 380, SEQ ID No. 426, SEQ ID No. 438, SEQ ID No. 448, SEQ ID No. 453, SEQ ID No. 455, SEQ ID No. 456, SEQ ID No. 458, SEQ ID No. 501, SEQ ID No. 516, SEQ ID No. 530, SEQ ID No. 542, SEQ ID No. 551, SEQ ID No. 720, SEQ ID No. 761, SEQ ID No. 762, SEQ ID No. 814, SEQ ID No. 859, SEQ ID No. 860, SEQ ID No. 861, SEQ ID No. 862, SEQ ID No. 869, SEQ ID No. 1079, SEQ ID No. 1168, SEQ ID No. 1174, SEQ ID No. 1176, SEQ ID No. 1413, SEQ ID No. 1414, SEQ ID No. 1415, SEQ ID No. 1416, SEQ ID No. 1417, SEQ ID No. 1457, SEQ ID No. 1651, SEQ ID No. 1856, SEQ ID No. 1869, SEQ ID No. 2021, SEQ ID No. 2080, SEQ ID No. 2152, SEQ ID No. 2162, SEQ ID No. 2173, SEQ ID No. 2251, SEQ ID No. 2295, SEQ ID No. 2306, SEQ ID No. 2317, SEQ ID No. 2328, SEQ ID No. 2340, SEQ ID No. 2342, SEQ ID No. 2351, SEQ ID No. 2500, SEQ ID No. 3228, SEQ ID No. 3230, SEQ ID No. 3311, SEQ ID No. 3317, SEQ ID No. 3318, SEQ ID No. 3319, SEQ ID No. 3320, SEQ ID No. 3322, SEQ ID No. 3323, SEQ ID No. 3326, SEQ ID No. 3327, SEQ ID No. 3328, SEQ ID No. 3375, SEQ ID No. 3376, SEQ ID No. 3377, SEQ ID No. 3378, SEQ ID No. 3422, SEQ ID No. 3489, SEQ ID No. 3503, SEQ ID No. 3609, SEQ ID No. 3623, SEQ ID No. 3624, SEQ ID No. 3772, SEQ ID No. 3783, SEQ ID No. 3788 and SEQ ID No. 3794; or
b) said polypeptide is encoded by a nucleotide sequence selected from the group consisting of SEQ ID No. 5835 to SEQ ID No. 10784.

64. A polypeptide, comprising a polypeptide selected from the group consisting of:
a) a polypeptide as claimed in claim 62;
b) a polypeptide exhibiting at least 80% identity with a polypeptide of sequence selected from the group consisting of SEQ ID No. 42 to SEQ ID No. 3855 or with a polypeptide encoded by a sequence selected from the group consisting of SEQ ID No. 5835 to SEQ ID No. 10784;
c) a fragment of at least 5 amino acids of a polypeptide of sequence selected from the group consisting of SEQ ID No. 42 to SEQ ID No. 3855 or with a polypeptide encoded by a sequence selected from the group consisting of SEQ ID No. 5835 to SEQ ID No. 10784;
d) a biologically active fragment of a polypeptide of sequence selected from the group consisting of SEQ ID No. 42 to SEQ ID No. 3855 or with a polypeptide encoded by a sequence selected from the group consisting of SEQ ID No. 5835 to SEQ ID No. 10784; and
e) a polypeptide of sequence selected from the group consisting of SEQ ID No. 42 to SEQ ID No. 3855 or with a polypeptide encoded by a sequence selected from the group consisting of SEQ ID No. 5835 to SEQ ID No. 10784, or as defined in c) or d), which has been modified.

65. A nucleotide sequence encoding a polypeptide as claimed in claim 62.

66. The nucleotide sequence as claimed in claim 58, wherein said nucleotide sequence encodes a polypeptide of P. luminescens having toxin activity or antibiotic activity, or is involved in the synthesis of toxins or antibiotics.

67. The polypeptide as claimed in claim 62, wherein said polypeptide is a polypeptide of P. luminescens having toxin activity or antibiotic activity, or is involved in the synthesis of toxins or antibiotics, or a fragment thereof.

68. A recording medium, comprising one or more nucleotide sequences as claimed in claim 57.

69. The recording medium as claimed in claim 68, wherein said recording medium is selected from the group consisting of a CD-ROM, a computer disk and a computer server.

70. A method of identifying primers or probes for determining genes in strains related to P. luminescens comprising
inspecting the sequences recorded on a recording medium as claimed in claim 68 and
identifying a primer pair or probe corresponding to a desired nucleotide or to a nucleotide sequence encoding a polypeptide with a desired function.

71. A method of studying the genetic polymorphism of strains related to P. luminescens comprising
obtaining a nucleotide sequence from a strain related to P. luminescens
sequencing the nucleotide sequence
comparing the nucleotide sequence to the nucleotide sequences recorded on a recording medium as claimed in claim 68.

72. A method for the automatic annotation of genes originating from a genome other than P. luminescens comprising
obtaining genomic DNA from a strain other than P. luminescens
sequencing the genomic DNA
comparing the nucleotide sequence of the genomic DNA to the nucleotide sequences recorded on a recording medium as claimed in claim 68 to assign putative function associated therewith.

73. A recording medium, comprising one or more polypeptides sequences as claimed in claim 62.

74. The recording medium as claimed in claim 73, wherein said recording medium is selected from the group consisting of a CD-ROM, a computer disk and a computer server.

75. A method of identifying primers or probes for determining genes in strains related to P. luminescens comprising
inspecting the sequences recorded on a recording medium as claimed in claim 73 and
identifying a primer pair or probe corresponding to a desired nucleotide or to a nucleotide sequence encoding a polypeptide with a desired function.

76. A method of studying the genetic polymorphism of strains related to P. luminescens comprising
obtaining a nucleotide sequence from a strain related to P. luminescens
sequencing the nucleotide sequence
comparing the nucleotide sequence to the nucleotide sequences encoding the polypeptide sequences recorded on a recording medium as claimed in claim 73.

77. A method for the automatic annotation of genes originating from a genome other than P. luminescens comprising
obtaining genomic DNA from a strain other than P. luminescens
sequencing the genomic DNA
comparing the nucleotide sequence of the genomic DNA to the nucleotide sequences encoding the polypeptide sequences recorded on a recording medium as claimed in claim 73 to assign putative function associated therewith

78. A primer or a probe, comprising one or more sequences as claimed in claim 57.

79. The nucleotide sequence as claimed in claim 78, wherein said primer or probe is labeled with a radioactive compound or with a nonradioactive compound.

80. The nucleotide sequence as claimed in claim 78, wherein said primer or probe is immobilized on a support by a covalent or noncovalent interaction.

81. The nucleotide sequence as claimed in claim 80, wherein said support is a high density filter or a DNA chip.

82. A DNA chip or a filter, comprising one or more nucleotide sequences as claimed in claim 78.

83. The DNA chip or the filter as claimed in claim 82, further comprising one or more nucleotide sequences from a cell from a source selected from the group consisting of a plant, an animal and a microorganism other than P. luminescens, wherein said nucleotide sequences are immobilized on the support of said chip.

84. The DNA chip or the filter as claimed in claim 83, wherein said cell is a cell or microorganism sensitive to a toxin or an antibiotic produced by P. luminescens, a bacterium of the genus Photorhabdus, and a variant of P. luminescens.

85. A kit for detecting and/or quantifying the expression of at least one gene of P. luminescens, comprising a DNA chip or a filter as claimed in claim 82.

86. A cloning and/or expression vector, comprising a nucleotide sequence as claimed in claim 57.

87. The cloning and/or expression vector as claimed in claim 86, comprising a nucleotide sequence selected from the group consisting of SEQ ID No. 3856 to SEQ ID No. 5825 and SEQ ID No. 5835 to SEQ ID No. 10784, or fragments thereof.

88. A host cell, transformed with a vector as claimed in claim 86.

89. A plant or an animal, except a human, comprising a transformed cell as claimed in claim 88.

90. A method for preparing a polypeptide, comprising
culturing a cell transformed with a vector as claimed in claim 88 under conditions which allow the expression of said polypeptide, and
recovering the resultant recombinant polypeptide.

91. A recombinant polypeptide obtained by the method as claimed in claim 90.

92. A method for preparing a polypeptide as claimed in claim 62, comprising
chemical synthesizing said polypeptide and
isolating said polypeptide.

93. An antibody selected from the group consisting of a monoclonal antibody, a polyclonal antibody, and a chimeric antibody, or a fragment thereof, wherein said antibody specifically recognizes a polypeptide as claimed in claim 62.

94. The antibody as claimed in claim 93, wherein said antibody is a labeled antibody.

95. A method for detecting and/or identifying bacteria belonging to the species P. luminescens, in a biological sample, comprising
a) contacting said biological sample with an antibody as claimed in claim 93;
b) detecting the formation of an antigen-antibody complex.

96. A method for detecting the expression of a gene of P. luminescens, comprising
contacting a strain of P. luminescens with an antibody as claimed in claim 93, and
detecting the formation of an antigen-antibody complex.

97. A kit for performing the method as claimed in claim 95, comprising the following elements:
a) said antibody; and at least one of either
b) reagents for constituting the medium suitable for an immunoreaction; or
c) reagents for detecting the antigen-antibody complexes resulting from the immunoreaction.

98. A kit for performing the method as claimed in claim 96, comprising the following elements:
a) said antibody;
b) reagents for constituting the medium suitable for an immunoreaction; and
c) reagents for detecting the antigen-antibody complexes resulting from the immunoreaction.

99. The polypeptide as claimed in claim 62, wherein said polypeptide is immobilized on a support

100. The polypeptide as claimed in claim 99, wherein said support a protein chip.

101. The polypeptide as claimed in claim 91, wherein said polypeptide is immobilized on a support

102. The polypeptide as claimed in claim 101, wherein said support a protein chip.

103. The antibody as claimed in claim 93, wherein said polypeptide is immobilized on a support

104. The antibody as claimed in claim 103, wherein said support a protein chip.

105. A protein chip comprising one or more polypeptide as claimed in claim 62 immobilized on the support of said chip.

106. The protein chip as claimed in claim 105, further comprising one or more polypeptides from a cell from a source selected from the group consisting of a plant, an animal and a microorganism other than P. luminescens, wherein said polypeptides are immobilized on the support of said chip.

107. The protein chip as claimed in claim 106, wherein said cell is a cell or microorganism sensitive to a toxin or an antibiotic produced by P. luminescens, a bacterium of the genus Photorhabdus, and a variant of P. luminescens.

108. A kit for detecting and/or quantifying the expression of at least one gene of P. luminescens, comprising a protein chip as claimed in claim 105.

109. A protein chip comprising one or more polypeptide as claimed in claim 91 immobilized on the support of said chip.

110. The protein chip as claimed in claim 109, further comprising one or more polypeptides from a cell from a source selected from the group consisting of a plant, an animal and a microorganism other than P. luminescens, wherein said polypeptides are immobilized on the support of said chip.

111. The protein chip as claimed in claim 110, wherein said cell is a cell or microorganism sensitive to a toxin or an antibiotic produced by P. luminescens, a bacterium of the genus Photorhabdus, and a variant of P. luminescens.

112. A kit for detecting and/or quantifying the expression of at least one gene of P. luminescens, comprising a protein chip as claimed in claim 109.

113. A protein chip comprising one or more antibodies as claimed in claim 93 immobilized on the support of said chip.

114. The protein chip as claimed in claim 113, further comprising one or more polypeptides from a cell from a source selected from the group consisting of a plant, an animal and a microorganism other than P. luminescens, wherein said polypeptides are immobilized on the support of said chip.

115. The protein chip as claimed in claim 114, wherein said cell is a cell or microorganism sensitive to a toxin or an antibiotic produced by P. luminescens, a bacterium of the genus Photorhabdus, and a variant of P. luminescens.

116. A kit for detecting and/or quantifying the expression of at least one gene of P. luminescens, comprising a protein chip as claimed in claim 113.

117. A method for detecting and/or identifying bacteria belonging to the species P. luminescens, in a biological sample, comprising:
a) isolating the DNA, or cDNA from the RNA, from the biological sample;
b) specifically amplifying the DNA of bacteria belonging to the species P. luminescens using at least one primer as claimed in claim 78;
c) identifying the amplification products.

118. A kit or set for detecting and/or identifying bacteria belonging to the species P. luminescens, comprising:
a) a nucleotide probe and/or primer as claimed in claim 78; and at least one of
b) reagents required for carrying out a hybridization reaction; or
c) reagents for a DNA amplification reaction.

119. A composition comprising one or more nucleotide sequences as claimed in claim 57.

120. A pharmaceutical composition comprising the composition as claimed in claim 119 and a pharmaceutically acceptable vehicle.

121. A biopesticidal composition comprising the composition as claimed in claim 119.

122. A composition comprising one or more polypeptides as claimed in claim 62.

123. A pharmaceutical composition comprising the composition as claimed in claim 122 and a pharmaceutically acceptable vehicle.

124. A biopesticidal composition comprising the composition as claimed in claim 122.

125. A composition comprising a vector as claimed in claim 86.

126. A pharmaceutical composition comprising the composition as claimed in claim 125 and a pharmaceutically acceptable vehicle.

127. A biopesticidal composition comprising the composition as claimed in claim 125.

128. A composition comprising one or more antibodies as claimed in claim 93.

129. A pharmaceutical composition comprising the composition as claimed in claim 128 and a pharmaceutically acceptable vehicle.

130. A biopesticidal composition comprising the composition as claimed in claim 128.

131. A method of preparing a toxin or an antibiotic comprising
culturing a cell as claimed in claim 88, and
expressing a polypeptide involved in production of a toxin or an antibiotic.

132. A genomic library of a bacterium of the genus Photorhabdus.

133. The genomic DNA library of a bacterium of the genus Photorhabdus as claimed in claim 132, wherein said DNA library is cloned into a plasmid.

134. The genomic DNA library as claimed in claim 132, wherein said bacterium is P. luminescens or P. luminescens strain TT01.

135. The genomic DNA library as claimed in claim 132, wherein said genomic DNA library is the genomic DNA library deposited with the CNCM on May 12, 2000, under accession No. I-2478.

136. A method for identifying at least one nucleotide sequence of P. luminescens not present in the genome of another species of bacterium or for identifying at least one nucleotide sequence of a genome of a bacterium of a species other than P. luminescens and not present in the P. luminescens genome, comprising:
a) aligning the genomic sequences of the other bacterial species with the nucleotide sequences of P. luminescens as claimed in claim 57; and
b) analyzing the data obtained by said aligning to identify and isolate said sequences only present in one or the other genome.

137. A method for identifying at least one nucleotide sequence of P. luminescens not present in the genome of another species of bacterium or for identifying at least one nucleotide sequence of a genome of a bacterium of a species other than P. luminescens and not present in the P. luminescens genome, comprising:
a) aligning the genomic sequences of the other bacterial species with the genomic DNA library as claimed in claim 134; and
b) analyzing the data obtained by said aligning to identify and isolate said sequences only present in one or the other genome.
